(19) United States
(12) Patent Application Publication
(10) Pub. No.: US 2024/0207425 A1
(43) Pub. Date: Jun. 27, 2024

(54) AURISTATIN LINKER-PAYLOADS, PHARMACEUTICAL COMPOSITIONS, AND USES THEREOF

(71) Applicant: Merck Sharp & Dohme LLC, Rahway, NJ (US)

(72) Inventors: Manoj B. Charati, Redwood City, CA (US); John A. Flygare, Burlingame, CA (US); Rebecca Elizabeth Johnson, Oakland, CA (US); Simon B. Lang, San Francisco, CA (US); W. Michael Seganish, Castro Valley, CA (US)

(73) Assignee: Merck Sharp & Dohme LLC, Rahway, NJ (US)

(21) Appl. No.: 18/536,350

(22) Filed: Dec. 12, 2023

Related U.S. Application Data
(60) Provisional application No. 63/497,887, filed on Apr. 24, 2023.
(60) Provisional application No. 63/432,470, filed on Dec. 14, 2022.

(51) Int. Cl. A61K 47/68; A61P 35/00; C07D 401/12; C07D 403/12

(52) U.S. Cl. CPC A61K 47/68031; A61P 35/00; C07D 401/12; C07D 403/12

(57) ABSTRACT

The present disclosure is directed to linker-payloads, and pharmaceutically acceptable salts, solvates, or stereoisomer thereof, comprising a structure of formula I:

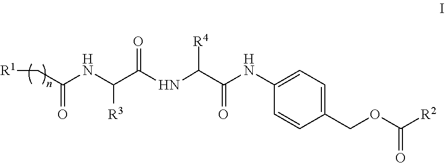

The disclosure is also directed to pharmaceutical compositions comprising these compounds and the use of these compounds, intermediates thereof, and compositions in the prevention or treatment of cancers and/or tumors.

### CROSS-REFERENCE TO RELATED APPLICATIONS

This application claims the benefit of priority to U.S. Provisional Application No. 63/432,470, filed Dec. 14, 2022, and U.S. Provisional Application No. 63/497,887, filed Apr. 24, 2023, the disclosures of which are incorporated herein by its entirety.

### BACKGROUND OF THE DISCLOSURE

The antibody-drug conjugate (ADC) is a therapeutic modality consisting of a monoclonal antibody attached to cytotoxic payloads and has made significant advances in serving as a transport vehicle that recognizes and binds to a protein antigen expressed in tumor tissues. The localized delivery and release of the payload within or near malignant cells allows for target delivery of a potent cytotoxic agent to disease tissue, while reducing damage to normal tissues. The linker component of ADCs is one important feature in developing optimized therapeutic agents that are highly active at well tolerated doses (see WO2021055865, WO2018025168 and US20130309256).

There remains a need for cytotoxin linker-payloads with utility for conjugation to antibodies or other targeting moieties to generate antibody-drug conjugates (ADCs), or other targeting ligand conjugates, particularly for oncology indications.

### SUMMARY OF THE DISCLOSURE

This disclosure provides linkers and linker-payloads with utility for conjugation to antibodies or other targeting moieties to generate Ligand-Drug Conjugates (e.g., ADCs), useful for oncology and other indications. Specifically, novel linker-payload structures are disclosed containing a maleimide or pyridyl sulfone attached to a cleavable dipeptide linker and a p-aminobenzyl carbamate (PABC) connection to a cytotoxic payload (drug) such as monomethylauristatin E (MMAE), monometylaurisatin F (MMAF), or other cytotoxic payloads as disclosed herein. Utility of these linker-payloads is demonstrated by conjugation to cysteine residues in a ligand (e.g., antibody, or an antigen-binding fragment of an antibody) to yield Ligand-Drug Conjugates (e.g., ADCs), which show favorable physical-chemical properties and high target-mediated potency. The linker-payloads are used to yield potent and novel Ligand-Drug Conjugates active across multiple cancer cell lines and demonstrate broad utility to be conjugated to several antibodies or other targeting moieties while still retaining favorable properties and efficacy. Thus, an aspect of this disclosure are Ligand-Drug Candidates having linker dipeptide sequences that provide enhanced exposure of released free cytotoxic compound to tumor tissue in comparison to normal tissue and thus selective entry into targeted cells and decreased exposure of normal tissue in comparison to tumor tissue to the cytotoxic compound.

Exemplary linker-payload compounds include, but are not limited to, the structures shown throughout this disclosure. Exemplary Ligand-Drug Conjugates (e.g, ADCs) formed using the linker-payloads described herein are also described. Other embodiments, aspects and features of the present disclosure are either further described in or will be apparent from the ensuing description, examples and appended claims.

In each of the embodiments described herein, each variable is selected independently of the other unless otherwise noted.

In one embodiment, the present disclosure provides a dipeptide linker-payload (drug), and pharmaceutically acceptable salts, solvates, and stereoisomers thereof, comprising a structure of Formula I:

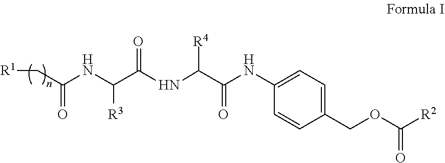

wherein:
R¹ is selected from:

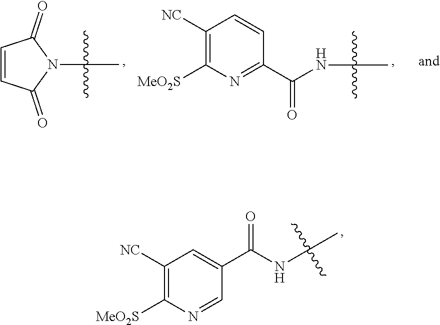 [SMILES: *N1C(=O)C=CC1=O ; *NC(=O)c1ccc(C#N)c(S(C)(=O)=O)n1 ; *NC(=O)c1cnc(S(C)(=O)=O)c(C#N)c1]

[symbol] wavy line indicates the site of covalent attachment;
R² is a cytotoxic drug;
R³ and R⁴ independently represent C₁₋₃ alkyl or a naturally occurring or unnatural amino acid side chain; and
n is an integer from 1 to 4.

An embodiment of Formula I is realized when n is 1.

An embodiment of Formula I is realized when n is 2.

An embodiment of Formula I is realized when n is 3.

An embodiment of Formula I is realized when n is 4.

An embodiment of Formula I is realized when R¹ is

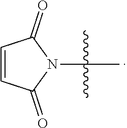 [SMILES: *N1C(=O)C=CC1=O]

Another embodiment of Formula I is realized when R¹ is

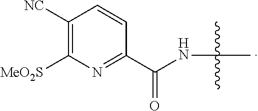 [SMILES: *NC(=O)c1ccc(C#N)c(S(C)(=O)=O)n1]

Another embodiment of Formula I is realized when R¹ is

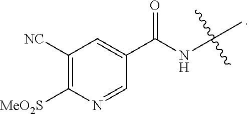 [SMILES: *NC(=O)c1cnc(S(C)(=O)=O)c(C#N)c1]

Another embodiment of Formula I is realized when R² is a cytotoxic drug or payload selected from anthracyclines, auristatins, camptothecins, duocarmycins, etoposides, maytansinoids, pyrrolobenzodiazepine dimers, DNA minor groove binders, taxanes, vinca alkaloids, enediynes, anti-tubulins, and vinca alkaloids. A subembodiment of this aspect of the disclosure is realized when R² is selected from auristatin T, auristatin E, auristatin F phenylenediamine, AEB, AEVB, monomethyl auristatin F (MMAF), lipophilic monomethyl aurstatin F, monomethyl auristatin E (MMAE)), lexitropsins, duocarmycins, paclitaxel and docetaxel, T67 (Tularik), vincristine, vinblastine, vindesine, vinorelbine, nicotinamide phosphoribosyltranferase inhibitor (NAMPTi), tubulysin M, doxorubicin, morpholino-doxorubicin, cyanomorpholino-doxorubicin, melphalan, methotrexate, mitomycin C, etoposide, CC-1065 analogue, calicheamicin, maytansine, an analog of dolastatin 10, rhizoxin, palytoxin, baccatin derivatives, taxane analogs (e.g., epothilone A and B), nocodazole, colchicine and colcimid, estramustine, cryptophysins, cemadotin, maytansinoids, combretastatins, discodermoide and eleuthrobin. The cytotoxic or cytostatic agent can be an anti-tubulin agent. Examples of anti-tubulin agents include taxanes (e.g., Taxol® (paclitaxel), Taxotere® (docetaxel)), T67 (Tularik), vinca alkyloids (e.g., Other suitable antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermoide and eleuthrobin.

Another subembodiment of this aspect of the disclosure is realized when R² is an auristatin drug. An aspect of this subembodiment is realized when R² is an auristatin drug selected from AE, auristatin F phenylenediamine (AFP), AEB, AEVB, MMAF, and MMAE. A further aspect of this embodiment is realized when R² is MMAE. A further aspect of this embodiment is realized when R² is MMAF.

Another embodiment of Formula I is realized when R² is a pyrrolobenzodiazepine dimer. A subembodiment of this aspect is realized when R² is tesirine.

An embodiment of Formula I is realized when R³ and R⁴ are independently selected from C₁₋₃ alkyl. A subembodiment of this aspect of Formula I is realized when R³ and R⁴ are independently selected from —CH₃, —CH₂CH₃, —(CH₂)₂CH₃. A subembodiment of this aspect of Formula I is realized when R³ and R⁴ are both CH₃. A subembodiment of this aspect of Formula I is realized when R³ and R⁴ are both —CH₂CH₃. A subembodiment of this aspect of Formula I is realized when R³ and R⁴ are both —(CH₂)₂CH₃. A subembodiment of this aspect of Formula I is realized when one of R³ and R⁴ is CH₃ and the other is selected from —CH₂CH₃, and —(CH₂)₂CH₃.

An embodiment of Formula I is realized when R³ and R⁴ are independently a naturally occurring or non-naturally occurring amino acid side chain. A subembodiment of this aspect of Formula I is realized when R³ and R⁴ are independently selected from arginine, glutamine, phenylalanine, tyrosine, tryptophan, lysine, glycine, alanine, histidine, serine, proline, glutamic acid, aspartic acid, threonine, cysteine, methionine, leucine, asparagine, isoleucine, and valine or side chains, in the L or D-configuration. Another embodiment of Formula I is realized when R³ and R⁴ are independently selected from a naturally occurring or non-naturally occurring amino acid side chain selected from the side chains of alanine, valine, leucine and methionine.

In another embodiment, the present disclosure provides a compound of Formula II and pharmaceutically acceptable salts, solvates, and stereoisomers thereof, comprising a structure of:

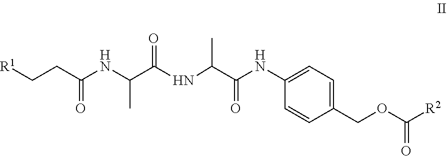

wherein R¹ and R² are as described herein. An embodiment of Formula II is realized when R² is an auristatin drug. An aspect of this subembodiment is realized when R² is an auristatin drug selected from AE, auristatin F phenylenediamine (AFP), AEB, AEVB, MMAF, and MMAE. A further aspect of Formula II is realized when R² is MMAE. A further aspect of Formula II is realized when R² is MMAF.

An embodiment of Formula II is realized when R¹ is

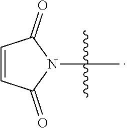 [SMILES: *N1C(=O)C=CC1=O]

An embodiment of Formula II is realized when R¹ is

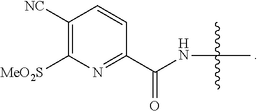 [SMILES: *NC(=O)c1ccc(C#N)c(S(C)(=O)=O)n1]

An embodiment of Formula II is realized when R¹ is

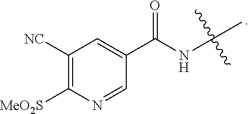 [SMILES: *NC(=O)c1cnc(S(C)(=O)=O)c(C#N)c1]

Another embodiment of Formula II is realized when R¹ is

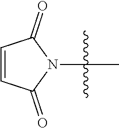 [SMILES: *N1C(=O)C=CC1=O]

### and R² is MMAE.

Another embodiment of Formula II is realized when R¹ is

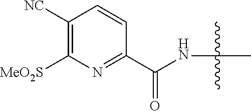 [SMILES: *NC(=O)c1ccc(C#N)c(S(C)(=O)=O)n1]

### and R² is MMAE.

Another embodiment of Formula II is realized when R¹ is

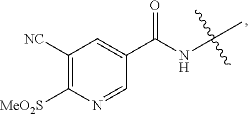 [SMILES: *NC(=O)c1cnc(S(C)(=O)=O)c(C#N)c1]

### and R² is MMAE.

Another embodiment of Formula II is realized when R² is a pyrrolobenzodiazepine dimer. A subembodiment of this aspect is realized when R² is tesirine.

Another embodiment of Formula II is represented by structural Formula II′:

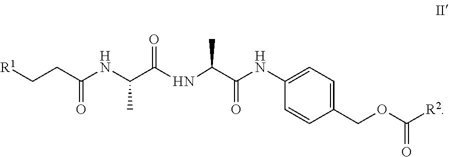

In another embodiment, the present disclosure provides a compound of Formula III and pharmaceutically acceptable salts, solvates, and stereoisomers thereof, comprising a structure of:

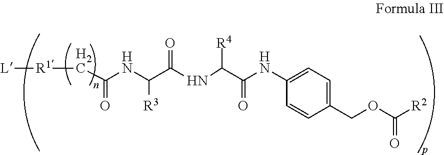

wherein L′ is a ligand that is an antibody, or an antigen-binding fragment of an antibody), p denotes the average number of drug linker moieties in a Ligand-Drug Conjugate composition and R², R³, and R⁴ are as described herein and R¹′ is selected from:

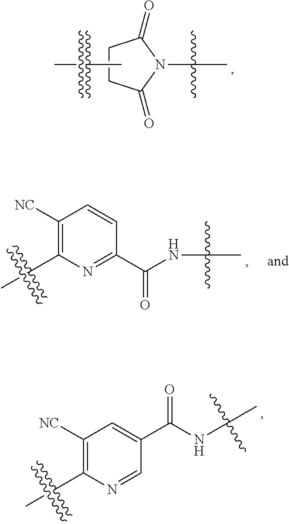

[symbol]

single wavy line indicates the site of covalent attachment to —(CH₂)ₙ; [symbol]
double wavy line indicates the site of covalent attachment to a sulfur of a cysteine residue of L′. It is understood that where L′ is an antibody, a sulfur atom S bonded to L′ in the Formula III represents a sulfur of the side chain of a cysteine side chain of the antibody. P is a positive real number, including all the fractions, and decimal values. In some embodiments, p is a positive rational number from 1 to 24, 1 to 12, 1 to 8, or is 4 or 8, which includes fractions and decimal values. In some embodiments, p is 1, 2, 3, 4, 5, 6, 7, 8, 9, 10, 11, 12, 13, 14, 15, 16, 17, 18, 19, 20, 21, 22, 23, and 24, or fraction and decimal values between 1 to 24. In some embodiments, p is 2, 4, 6, or 8, or p is 1 to 2, including fraction and decimal values between 2 to 8.

Another embodiment of Formula III is represented by structural Formula III′ or III″:

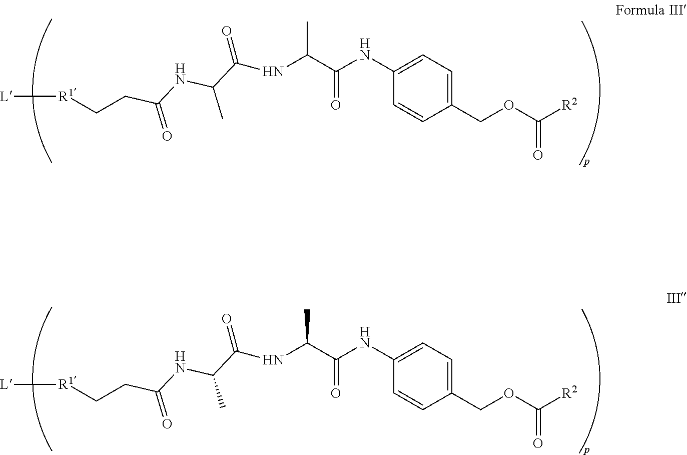

wherein L′, R¹′, and R² are as described herein.

An embodiment of Formula III, III′ and III″ is realized when L′ includes intact monoclonal antibodies, polyclonal antibodies, monospecific antibodies, multi-specific antibodies (e.g., bispecfic antibodies), and antibody fragments that exhibit the desired biological activity. It is further envisioned that the antibody drug conjugates of this disclosure might be replaced with anything that specifically binds or reactively associates or complexes with a receptor, antigen or other receptive moiety associated with a given target-cell population. For example, instead of containing an antibody, conjugates of the disclosure might contain a targeting molecule that binds to complexes with, or reacts with a receptor, antigen or other receptive moiety of a cell population sought to be therapeutically or otherwise biologically modified (e.g., small molecular weight proteins, polypeptide or peptides, lectins, glycoproteins, non-peptides, vitamins, nutrient-transport molecules (e.g., transferrin)) and any other cell binding molecule or substance.

Examples of ligand L′ useful in the present disclosure are disclosed herein selected from trastuzumab, or mutants thereof, oregovomab, edrecolomab, cetuximab, a humanized monoclonal antibody to the vitronectin receptor (aᵥb₃), alemtuzumab, anti-HLA-DR antibodies including a humanized anti-HLA-DR antibody for the treatment of non-Hodgkin's lymphoma, 121I Lym-1, anti-HLA-Dr10 antibodies including a murine anti-DLA-Dr10 antibody for the treatment of non-Hodgkin's lymphoma, anti-CD33 antibodies, anti-CD22 antibodies including a humanized anti-CD22 mAb for the treatment of Hodgkin's Disease or non-Hodgkin's lymphoma, labetuzumab, bevacizumab, ibritumomab tiuxetan, ofatumumab, panitumumab, rituximab, tositumamab, ipilimumab, sacituzumab, cetuximab (ERBITUX), and gentuzumab.

An aspect of this disclosure relates to a pharmaceutical composition comprising a therapeutically effective amount of a compound of Formula I, II, II′, III, III′, III″ or a pharmaceutically acceptable salt or solvate thereof and one or more pharmaceutically acceptable carrier(s), diluent(s) or excipients(s).

Another aspect of the disclosure relates to a pharmaceutical composition comprising a therapeutically effective amount of a compound of Formula I, II, II′, III, III′, or III″ as described herein, or a tautomer, mesomere, racemate, enantiomer, diastereomer thereof, or mixture thereof, or a pharmaceutically acceptable salt thereof and one or more pharmaceutically acceptable carrier(s), diluent(s) or excipient(s).

Another aspect of the disclosure relates to a compound of Formula I, II, II′, III, III′, or III″ as described herein, or a tautomer, mesomere, racemate, enantiomer, diastereomer thereof, or mixture thereof, or a pharmaceutically acceptable salt thereof for use as a drug or drug component.

Another aspect of the disclosure relates to a compound of Formula I, II, II′, III, III′, or III″ as described herein, or a tautomer, mesomere, racemate, enantiomer, diastereomer thereof, or mixture thereof, or a pharmaceutically acceptable salt thereof, or a pharmaceutical composition in the preparation of a medicament for treating or preventing a tumor.

In another embodiment, the compounds of the disclosure include those identified herein as Examples in the tables below, and pharmaceutically acceptable salts thereof.

### DETAILED DESCRIPTION OF THE DISCLOSURE

For each of the following embodiments, any variable not explicitly defined in the embodiment is as defined in Formula (I). In each of the embodiments described herein, each variable is selected independently of the other unless otherwise noted.

The compounds of the disclosure may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all the possible optical isomers and diastereomers in mixtures and as pure or partially purified compounds are included within the ambit of this disclosure. Unless a specific stereochemistry is indicated, the present disclosure is meant to encompass all such isomeric forms of these compounds.

The independent syntheses of these diastereomers or their chromatographic separations may be achieved as known in the art by appropriate modification of the methodology disclosed herein. Their absolute stereochemistry may be determined, amongst other methods, by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

In the compounds of Formulae I, II, II′, III, III′, or III″ the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure may include all suitable isotopic variations of the compounds of generic Formulae I, II, II′, III, III′, or III″. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. For purposes of this disclosure when a compound is said to be “not deuterated” it means not enriched in deuterium beyond the background state. Isotopically-enriched compounds within generic Formulae I, II, II′, III, III′, or III″ can be prepared without undue experimentation by conventional techniques well known to those skilled in the art or by processes analogous to those described in the Schemes and Examples herein using appropriate isotopically-enriched reagents and/or intermediates.

When a compound of the disclosure can form tautomers, all such tautomeric forms are also included within the scope of the present disclosure. For example, compounds including carbonyl —CH₂C(O)— groups (keto forms) may undergo tautomerism to form hydroxyl —CH═C(OH)— groups (enol forms). Both keto and enol forms, where present, are included within the scope of the present disclosure.

When any variable (e.g., R⁵, etc.) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is bicyclic, it is intended that the bond be attached to any of the suitable atoms on either ring of the bicyclic moiety.

It is understood that one or more silicon (Si) atoms can be incorporated into the compounds of the instant disclosure in place of one or more carbon atoms by one of ordinary skill in the art to provide compounds that are chemically stable and that can be readily synthesized by techniques known in the art from readily available starting materials. Carbon and silicon differ in their covalent radius leading to differences in bond distance and the steric arrangement when comparing analogous C-clement and Si-clement bonds. These differences lead to subtle changes in the size and shape of silicon-containing compounds when compared to carbon. One of ordinary skill in the art would understand that size and shape differences can lead to subtle or dramatic changes in potency, solubility, lack of off-target activity, packaging properties, and so on. (Diass, J. O. et al. Organometallics (2006) 5:1188-1198; Showell, G. A. et al. Bioorganic & Medicinal Chemistry Letters (2006) 16:2555-2558).

It is understood that substituents and substitution patterns on the compounds of the instant disclosure can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be understood as meaning that the group in question is either unsubstituted or may be substituted with one or more substituents.

Absolute stereochemistry is illustrated by the use of hashed and solid wedge bonds. As shown in Illus-I and Illus-II. Accordingly, the methyl group of Illus-I is emerging from the page of the paper and the ethyl group in Illus-II is descending into the page, where the cyclohexene ring resides within the plane of the paper. It is assumed that the hydrogen on the same carbon as the methyl group of Illus-I descends into the page and the hydrogen on the same carbon as the ethyl group of Illus-II emerges from the page. The convention is the same where both a hashed and solid rectangle are appended to the same carbon as in Illus-III, the methyl group is emerging from the plane of the paper and the ethyl group is descending into the plane of the paper with the cyclohexene ring in the plane of the paper.

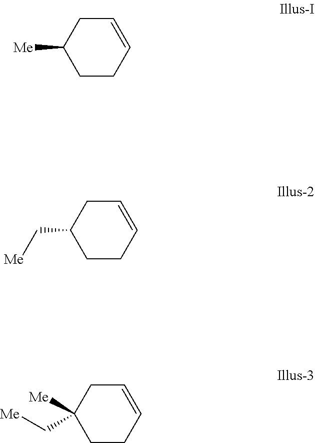 [SMILES: C[C@H]1CC=CCC1 ; CC[C@@H]1CC=CCC1 ; CC[C@]1(C)CC=CCC1]

As is conventional, unless otherwise noted in accompanying text, ordinary “stick” bonds or “wavy” bonds indicate that all possible stereochemistry is represented, including, pure compounds, mixtures of isomers, and racemic mixtures.

As used herein, unless otherwise specified, the following terms have the following meanings:

The phrase “at least one” used in reference to the number of components comprising a composition, for example, “at least one pharmaceutical excipient” means that one member of the specified group is present in the composition, and more than one may additionally be present. Components of a composition are typically aliquots of isolated pure material added to the composition, where the purity level of the isolated material added into the composition is the normally accepted purity level for a reagent of the type.

Whether used in reference to a substituent on a compound or a component of a pharmaceutical composition the phrase “one or more”, means the same as “at least one”;

“Effective amount” or “therapeutically effective amount” is meant to describe the provision of an amount of at least one compound of the disclosure or of a composition comprising at least one compound of the disclosure which is effective in treating or inhibiting a disease or condition described herein, and thus produce the desired therapeutic, ameliorative, inhibitory or preventative effect. For example, in treating central nervous system diseases or disorders with one or more of the compounds described herein “effective amount” (or “therapeutically effective amount”) means, for example, providing the amount of at least one compound of Formula I, Formula II, or Formula III that results in a therapeutic response in a patient afflicted with a central nervous system disease or disorder (“condition”), including a response suitable to manage, alleviate, ameliorate, or treat the condition or alleviate, ameliorate, reduce, or eradicate one or more symptoms attributed to the condition and/or long-term stabilization of the condition, for example, as may be determined by the analysis of pharmacodynamic markers or clinical evaluation of patients afflicted with the condition;

“Patient” and “subject” means an animal, such as a mammal (e.g., a human being) and is preferably a human being;

“Prodrug” means compounds that are rapidly transformed, for example, by hydrolysis in blood, in vivo to the parent compound, e.g., conversion of a prodrug of Formula I, Formula II, or Formula III to a compound of Formula I, Formula II, or Formula III or to a salt thereof; a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference; the scope of this disclosure includes prodrugs of the novel compounds of this disclosure;

The term “substituted” means that one or more of the enumerated substituents can occupy one or more of the bonding positions on the substrate typically occupied by “—H”, provided that such substitution does not exceed the normal valency rules for the atom in the bonding configuration presented in the substrate, and that the substitution ultimately provides a stable compound, which is to say that such substitution does not provide compounds with mutually reactive substituents located geminal or vicinal to each other; and wherein the substitution provides a compound sufficiently robust to survive isolation to a useful degree of purity from a reaction mixture.

Where optional substitution of a moiety is described (e.g., “optionally substituted”) the term means that if substituents are present, one or more of the enumerated substituents for the specified substrate can be present on the substrate in a bonding position normally occupied by the default substituent normally occupying that position. For example, a default substituent on the carbon atoms of an alkyl moiety is a hydrogen atom, an optional substituent can replace the default substituent.

As used herein, unless otherwise specified, the following terms used to describe moieties, whether comprising the entire definition of a variable portion of a structural representation of a compound of the disclosure or a substituent appended to a variable portion of a structural representation of a group of compounds of the disclosure have the following meanings, and unless otherwise specified, the definitions of each term (i.e., moiety or substituent) apply when that term is used individually or as a component of another term (e.g., the definition of aryl is the same for aryl and for the aryl portion of arylalkyl, alkylaryl, arylalkynyl moieties, and the like); moieties are equivalently described herein by structure, typographical representation or chemical terminology without intending any differentiation in meaning, for example, an “acyl” substituent may be equivalently described herein by the term “acyl”, by typographical representations “R′—(C═O)—” or “R′—C(O)—”, or by a structural representation:

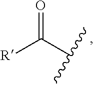

equally, with no differentiation implied using any or all of these representations.

The term “alkyl” (including the alkyl portions of other moieties, such as trifluoromethyl-alkyl- and alkoxy-) means a straight or branched aliphatic hydrocarbon moiety comprising up to about 20 carbon atoms (for example, a designation of “C₁₋₂₀ -alkyl” indicates an aliphatic hydrocarbon moiety of from 1 to 20 carbon atoms). In some embodiments, alkyls preferably comprise up to about 10 carbon atoms, unless the term is modified by an indication that a shorter chain is contemplated, for example, an alkyl moiety of from 1 up to 8 carbon atoms is designated herein “C₁₋₈-alkyl”. Where the term “alkyl” is indicated with two hyphens (i.e., “-alkyl-” it indicates that the alkyl moiety is bonded in a manner that the alkyl moiety connects the substituents on either side of it, for example, “-alkyl-OH” indicates an alkyl moiety connecting a hydroxyl moiety to a substrate.

As used herein, when the term “alkyl” is modified by “substituted” or “optionally substituted”, it means that one or more C—H bonds in the alkyl moiety group is substituted, or optionally may be substituted, by a substituent bonded to the alkyl substrate which is called out in defining the moiety.

The term “solvate” refers to a pharmaceutically acceptable solvate formed by a compound of the present disclosure with one or more solvent molecule(s). Non-limiting examples of solvent molecules include water, ethanol, acetonitrile, isopropanol, DMSO, ethyl acetate.

The term “halogen” means fluorine, chlorine, bromine, or iodine; preferred halogens, unless specified otherwise where the term is used, are fluorine, chlorine and bromine, a substituent which is a halogen atom means —F, —Cl, —Br, or —I, and “halo” means fluoro, chloro, bromo, or iodo substituents bonded to the moiety defined, for example, “haloalkyl” means an alkyl, as defined above, wherein one or more of the bonding positions on the alkyl moiety typically occupied by hydrogen atoms are instead occupied by a halo group, perhaloalkyl (or “fully halogenated” alkyl) means that all bonding positions not participating in bonding the alkyl substituent to a substrate are occupied by a halogen, for example, where the alkyl is selected to be methyl, the term perfluoroalkyl means —CF₃;

The term “hydroxyl” and “hydroxy” means an HO— group, “hydroxyalkyl” means a substituent of the formula: “HO-alkyl-”, wherein the alkyl group is bonded to the substrate and may be substituted or unsubstituted as defined above; preferred hydroxyalkyl moieties comprise a lower alkyl; Non-limiting examples of suitable hydroxyalkyl groups include hydroxymethyl and 2-hydroxyethyl.

The bonding sequence is indicated by hyphens where moieties are represented in text, for example -alkyl, indicates a single bond between a substrate and an alkyl moiety, -alkyl-X, indicates that an alkyl group bonds an “X” substituent to a substrate, and in structural representation, bonding sequence is indicated by a wavy line terminating a bond representation, for example:

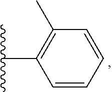 [SMILES: *c1ccccc1C]

indicates that the methylphenyl moiety is bonded to a substrate through a carbon atom ortho to the methyl substituent, while a bond representation terminated with a wavy line and drawn into a structure without any particular indication of an atom to which it is bonded indicates that the moiety may be bonded to a substrate via any of the atoms in the moiety which are available for bonding as described in the examples above.

The line —, as a bond generally indicates a mixture of, or either of, the possible isomers, e.g., containing (R)- and (S)-stereochemical configuration.

The term “DAR” or “Drug Antibody Ratio,” as used herein, refers to the average number of linker/drug moieties attached to the antibodies present in a composition. For a composition comprising an Antibody-Drug Conjugate of the present disclosure, the DAR for the composition is the average of the “p” of all of the individual Antibody-Drug Conjugate molecules present in said composition, and this average is expressed as a decimal. As such, in some embodiments for a composition comprising an Antibody-Drug Conjugate of the present disclosure, the DAR of the composition is a decimal from 0 to 24, 0 to 8, from 0 to 7, from 0 to 6, from 0 to 5, from 0 to 4, from 0 to 3, from 0 to 2, and from 0 to 1. In additional embodiments, for a composition comprising an Antibody-Drug Conjugate of the present disclosure, the DAR of the composition is a decimal from 1 to 4, 2 to 5, 3 to 6, 4 to 7, 5 to 8, and 6 to 8. In other embodiments, for a composition comprising an Antibody-Drug Conjugate of the present disclosure, the DAR of the composition is a decimal from 1 to 3, 2 to 4, 3 to 5, 4 to 6, 5 to 7, and 6 to 8. In further embodiments, for a composition comprising an Antibody-Drug Conjugate of the present disclosure, the DAR of the composition is a decimal from 1 to 2, 2 to 3, 3 to 4, 4 to 5, 5 to 6, 6 to 7, and 7 to 8. In particular embodiments, the DAR of the composition is 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, and 8.0. The term “composition” as used above, is understood to encompass pharmaceutical compositions. Average DAR can be determined by various conventional means such as UV spectroscopy, mass spectroscopy, ELISA assay, radiometric methods, hydrophobic interaction chromatography (HIC), electrophoresis and HPLC.

In all cases, compound name(s) accompany the structure drawn and are intended to capture each of the stereochemical permutations that are possible for a given structural isomer based on the synthetic operations employed in its preparation. Lists of discrete stereoisomers that are conjoined using or indicate that the presented compound (e.g. ‘Example number’) was isolated as a single stereoisomer, and that the identity of that stereoisomer corresponds to one of the possible configurations listed. Lists of discrete stereoisomers that are conjoined using and indicate that the presented compound was isolated as a racemic mixture or diastereomeric mixture.

“Natural amino acid” as used herein, unless otherwise stated or implied by context, refers to a naturally occurring amino acid, namely, arginine, glutamine, phenylalanine, tyrosine, tryptophan, lysine, glycine, alanine, histidine, serine, proline, glutamic acid, aspartic acid, threonine, cysteine, methionine, leucine, asparagine, isoleucine, and valine or a residue thereof, in the L or D-configuration, unless otherwise specified or implied by context.

“Unnatural amino acid” as used herein, unless otherwise stated or implied by context, refers to an alpha-amino-containing acid or residue thereof, which has the backbone structure of a natural amino acid, but for example has a side chain group attached to the alpha carbon that is not present in natural amino acids. Examples of unnatural amino acids are A′, B′, C′, D′ and E′ with amino acid side chains having structures:

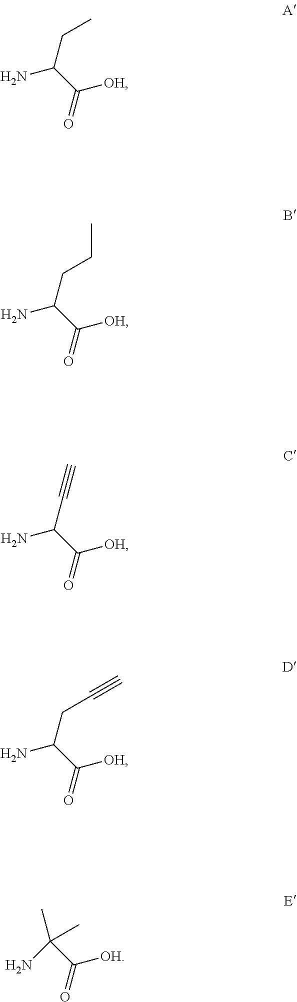

Examples of amino acid side chains are C₁₋₆ alkyl, C₂₋₆ alkenyl, and C₂₋₆ alkynyl. Further examples of amino acid side chains are di-methyl, ethyl, propyl, butyl, —C≡C, and —CH₂C≡C.

The ligand (L′) can be any moiety with a free cysteine group including, but not limited to, antibodies, proteins, peptides, polypeptides, or engineered antibodies modified to provide a free cysteine. An aspect of this is realized when the ligand is an antibody, preferably an intact antibody. The ligand acts to target and present the drug to the particular target cell population with which the ligand interacts. Suitable ligands include, for example, antibodies, e.g., full-length antibodies and antigen binding fragments thereof, interferons, lymphokines, hormones, growth factors and colony-stimulating factors, vitamins, nutrient transport molecules (such as, but not limited to, transferrin), or any other cell binding molecule or substance, including small molecules and peptides. The ligand can be, for example, a non-antibody protein targeting agent.

When the conjugates comprise non-immunoreactive protein, polypeptide, or peptide ligands instead of an antibody, useful non-immunoreactive protein, polypeptide, or peptide ligands include, but are not limited to, transferrin, epidermal growth factors (“EGF”), bombesin, gastrin, gastrin releasing peptide, platelet-derived growth factor, IL-2, IL-6, transforming growth factors (“TGF”), such as TGF-α and TGF-β, vaccinia growth factor (“VGF”), insulin and insulin-like growth factors I and II, somatostatin, lectins and apoprotein from low density lipoprotein.

Particularly preferred ligands (L′) are antibodies, including intact antibodies. In fact, in any of the embodiments described herein, the ligand can be an antibody. Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as for example, those having a variable region derived from a murine monoclonal and human immunoglobulin constant regions. (See, e.g., U.S. Pat. Nos. 4,816,567; and 4,816,397, which are incorporated herein by reference in their entirety.) Humanized antibodies are antibody molecules from non-human species having one or more complementarity determining regions (CDRs) from the non-human species and a framework region from a human immunoglobulin molecule. (See, e.g., U.S. Pat. No. 5,585,089, which is incorporated herein by reference in its entirety.) Such chimeric and humanized monoclonal antibodies can be produced by recombinant DNA techniques known in the art, for example using methods described in International Publication No. WO 87/02671; European Patent Publication No. 0 184 187each of which is incorporated herein by reference in its entirety.

Completely human antibodies are particularly desirable and can be produced using transgenic mice that are incapable of expressing endogenous immunoglobulin heavy and light chains genes, but which can express human heavy and light chain genes.

Antibodies include analogs and derivatives that are either modified, i.e., by the covalent attachment of any type of molecule as long as such covalent attachment permits the antibody to retain its antigen binding immunospecificity. For example, but not by way of limitation, derivatives and analogs of the antibodies include those that have been further modified, e.g., by glycosylation, acetylation, PEGylation, phosphorylation, amidation, derivatization by known protecting/blocking groups, proteolytic cleavage, linkage to a cellular antibody or other protein, etc. Any of numerous chemical modifications can be carried out by known techniques including, but not limited to, specific chemical cleavage, acetylation, formylation, metabolic synthesis in the presence of tunicamycin, etc. Additionally, the analog or derivative can contain one or more unnatural amino acids.

In a specific embodiment, known antibodies for the treatment of cancer can be used. Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing.

In another specific embodiment, antibodies for the treatment of an autoimmune disease are used in accordance with the compositions and methods of the disclosure. Antibodies immunospecific for an antigen of a cell that is responsible for producing autoimmune antibodies can be obtained from any organization (e.g., a university scientist or a company) or produced by any method known to one of skill in the art such as, e.g., chemical synthesis or recombinant expression techniques.

In another embodiment, it may be desirable to conjugate components of the linker to the ligand (e.g., antibody) prior to attaching the payload or drug component of the ADC. For example, in embodiments where a thiol containing substituent, e.g., cysteine, is being used to attach the drug component, it may be desirable to conjugate components of the linker to the ligand L′ (e.g., antibody) prior to attaching the drug component of the ADC.

In another embodiment, the compounds of the disclosure include those identified herein as Examples in the tables below, and pharmaceutically acceptable salts thereof.

“Antibody Drug Conjugate” or ADC, as the term is used herein, unless otherwise stated or implied by context, is a subset of Ligand-Drug Conjugates of Formula III, Formula III′ and Formula III″ therefore refers to a construct comprised of an antibody Ligand (L′) incorporating or corresponding to an antibody or antigen-binding fragment thereof, and a cytotoxic payload incorporating or corresponding in structure to a biologically active compound, often referred to as free drug, wherein L′ and R² herein are bonded to each other through a dipeptide linker Unit, wherein the Antibody-Drug Conjugate is capable of selective binding to a targeted antigen of a targeted cell, which in some aspects is an antigen of an abnormal cell such as a cancer cell, through its targeting antibody Ligand Unit.

The term Antibody-Drug Conjugate (ADC), in one aspect, refers to a plurality (i.e., composition) of individual Conjugate compounds having the same or differing to some extent by the number of linker-payload moieties conjugated to each antibody Ligand (L′) and/or the locations on the antibody Ligand (L′) to which the linker-payload moieties are conjugated. In some aspects the term refers to a distribution or collection (i.e., population or plurality) of Conjugate compounds having the same linker-payload moieties and antibody Ligands (L′), allowing for mutational amino acid variations and varying glycosylation patterns as described herein occurring during production of antibodies from cell culture, which in some aspects have variable loading and/or distribution of the linker-payload moieties attached to each antibody residue (as, for example, when the number of linker-payload moieties of any two Antibody-Drug Conjugate compounds in a plurality of such compounds is the same but the locations of their sites of attachment of the linker-payload moieties to the targeting antibody Ligand (L′) differ). In those instances, an Antibody-Drug Conjugate is described by the averaged drug loading of the Conjugate compounds.

In some aspects the payload (drug) is a cytotoxic agent, typically one that has a secondary aliphatic amine as the conjugation handle, and includes auristatin compounds as defined herein. Useful classes of cytotoxic agents include, for example, antitubulin agents, DNA minor groove binding agents, DNA replication inhibitors, chemotherapy sensitizers, or the like. Other exemplary classes of cytotoxic agents include anthracyclines, auristatins, camptothecins, duocarmycins, etoposides, maytansinoids and vinca alkaloids. Exemplary cytotoxic agents include for example, auristatin T, auristatin E, auristatin F phenylenediamine (AFP), monomethyl auristatin F (MMAF), lipophilic monomethyl aurstatin F, monomethyl auristatin E (MMAE)), DNA minor groove binders (e.g., enediynes and lexitropsins), duocarmycins, taxanes (e.g., paclitaxel and docetaxel), vinca alkaloids, nicotinamide phosphoribosyltranferase inhibitor (NAMPTi), tubulysin M, doxorubicin, morpholino-doxorubicin, and cyanomorpholino-doxorubicin.

The cytotoxic agent can be a chemotherapeutic such as, for example, doxorubicin, paclitaxel, melphalan, vinca alkaloids, methotrexate, mitomycin C or etoposide. The agent can also be a CC-1065 analogue, calicheamicin, maytansine, an analog of dolastatin 10, rhizoxin, or palytoxin.

Exemplary auristatins include, but are not limited to auristatin E (AE), reacting auristatin E with paraacetyl benzoic acid or benzoylvaleric acid to produce benzolyl-aurstatin E ester (AEB) and 5-benzoylvaleric acid-aurstatin E ester (AEVB), respectively, auristatin F phenylenediamine (AFP), monomethyl auristatin F (MMAF), and monomethyl auristatin E (MMAE) and those further described in the embodiments of the disclosure. The synthesis and structure of auristatins are described in U.S. Patent Application Publication Nos. 2003-0083263, 2005-0238649 2005-0009751, 2009-0111756, and 2011-0020343: International Patent Publication No. WO 04/010957, International Patent Publication No. WO 02/088172, and U.S. Pat. Nos. 7,659,241 and 8,343,928. Their structures and methods of their syntheses disclosed therein are specifically incorporated by reference herein.

The cytotoxic agent can be a DNA minor groove binding agent. (See, e.g., U.S. Pat. No. 6,130,237.) For example, the minor groove binding agent can be a CBI compound or an enediyne (e.g., calicheamicin).

The cytotoxic or cytostatic agent can be an anti-tubulin agent. Examples of anti-tubulin agents include taxanes (e.g., Taxol® (paclitaxel), Taxotere® (docetaxel)), T67 (Tularik), vinca alkyloids (e.g., vincristine, vinblastine, vindesine, and vinorelbine. Other suitable antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermoide and eleuthrobin.

The cytotoxic agent can be a maytansinoid, another group of anti-tubulin agents (e.g., DM1, DM2, DM3, DM4). For example, the maytansinoid can be maytansine or a maytansine containing drug linker such as DM-1 or DM-4 (ImmunoGen, Inc.; see also Chari et al., 1992, Cancer Res.)

Unsatisfied valences in the text, schemes, examples, structural formulae, and any Tables herein is assumed to have a hydrogen atom or atoms of sufficient number to satisfy the valences.

One or more compounds of the disclosure may also exist as, or optionally be converted to, a solvate. Preparation of solvates is generally known. Thus, for example, M. Caira et al., J. Pharmaceutical Sci., 93 (3), 601-611 (2004) describe the preparation of the solvates of the antifungal fluconazole in ethyl acetate as well as from water. Similar preparations of solvates, and hemisolvate, including hydrates (where the solvent is water or aqueous-based) and the like are described by E. C. van Tonder et al., AAPS PharmSciTech., 5 (1), article 12 (2004); and A. L. Bingham et al., Chem. Commun., 603-604 (2001). A typical, non-limiting, process involves dissolving the inventive compound in desired amounts of the desired solvent (for example, an organic solvent, an aqueous solvent, water or mixtures of two or more thereof) at a higher than ambient temperature, and cooling the solution, with or without an antisolvent present, at a rate sufficient to form crystals which are then isolated by standard methods. Analytical techniques such as, for example I.R. spectroscopy, show the presence of the solvent (including water) in the crystals as a solvate (or hydrate in the case where water is incorporated into the crystalline form).

This disclosure also includes the compounds of this disclosure in isolated and purified form obtained by routine techniques. Polymorphic forms of the compounds of Formula I, Formula II, and Formula II and of the salts, solvates and prodrugs of the compounds of Formula I, Formula II, and Formula II are intended to be included in the present disclosure. Certain compounds of the disclosure may exist in different isomeric forms (e.g., enantiomers, diastereoisomers, atropisomers). The inventive compounds include all isomeric forms thereof, both in pure form and admixtures of two or more, including racemic mixtures.

In the same manner, unless indicated otherwise, presenting a structural representation of any tautomeric form of a compound which exhibits tautomerism is meant to include all such tautomeric forms of the compound. Accordingly, where compounds of the disclosure, their salts, and solvates and prodrugs thereof, may exist in different tautomeric forms or in equilibrium among such forms, all such forms of the compound are embraced by, and included within the scope of the disclosure. Examples of such tautomers include, but are not limited to, ketone/enol tautomeric forms, imine-enamine tautomeric forms, and for example heteroaromatic forms such as the following moieties:

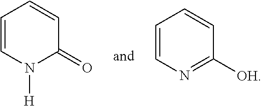 [SMILES: O=c1cccc[nH]1 ; Oc1ccccn1]

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives wherein the parent compound is modified by making acid or base salts thereof. Salts in the solid form may exist in more than one crystal structure and may also be in the form of hydrates. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as formic, hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Salts derived from inorganic bases include aluminum, ammonium, calcium, copper, ferric, ferrous, lithium, magnesium, manganic salts, manganous, potassium, sodium, zinc, and the like.

When the compound of the present disclosure is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In one aspect of the disclosure the salts are citric, hydrobromic, hydrochloric, maleic, phosphoric, sulfuric, fumaric, and tartaric acids. Similarly, the salts of the acidic compounds are formed by reactions with the appropriate inorganic or organic base.

The term “cytotoxic drug” refers to a substance that inhibits or stops the function of cells and/or causes cell death or destruction. Toxic drugs include toxins and other compounds that can be used in tumor treatment.

The term “toxin” refers to any substance that can have a harmful effect on the growth or proliferation of cells. Toxins can be small molecule toxins and their derivatives from bacteria, fungi, plants or animals, including Camptothecin derivatives such as exatecan, maytansinoid and its derivatives (CN101573384) such as DM1, DM3, DM4, auristatin F (AF) and its derivatives such as MMAF, MMAE, 3024 (WO 2016/127790 A1, compound 7), diphtheria toxin, exotoxin, ricin A chain, abrin A chain, modeccin, a-sarcin, Aleutites fordii toxic protein, dianthin toxic protein, Phytolaca americana toxic protein, Momordica charantia inhibitor, curcin, crotin, Sapaonaria ojficinalis inhibitor, gelonin, mitogellin, restrictocin, phenomycin, enomycin and trichothecenes.

The term “chemotherapeutic drug” refers to a chemical compound that can be used to treat tumors. This definition also includes antihormonal agents that act to modulate, reduce, block, or inhibit the effects of hormones that promote cancer growth, which are often in the form of systemic or holistic therapy. They can be hormones. Examples of chemotherapeutic drugs include alkylating agents, such as thiotepa; cyclosphamide (CYTOXAN™); alkyl sulfonate such as busulfan, improsulfan and piposul-fan; aziridine such as benaodopa, carboquone, meturedopa and uredopa; aziridine and methylamelamine including altretamine, triethy lenemelamine, triethy lenephosphor-amide, triethylenethiophosphoramide and trimethylolomela-mine; nitrogen mustards such as chlorambucil, chlornaphaz-ine, cholophosphamide, estramustine, ifosfamide, mechlorethamine, nitrobin hydrochloride; melphalan, novembichin, phenesterine, prednimustine, trofosfamide, uramustine; nitrosureas such as carmustine, chlorozotocin, fotemustine, lomustine, nimustine, ranimustine; antibiotic such as aclacinomycin, actinomycin, authramycin, azaser-ine, bleomycin, cactinomycin C, calicheamicin, carabicin, chromomycin, carzinophilin, chromomycin, actinomycin D, daunorubicin, detorubicin, 6-diazo-5-oxy-L-norleucine, doxorubicin, epirubicin, esorubicin, idarubicin, marcello-mycin, mitomycin, mycophenolic acid, nogalamycin, olivo-mycin, peplomycin, potfiromycin, puromycin, quelamycin, rodorubicin, streptonigrin; streptozocin, tuberculocidin, ubenimex, zinostatin, zorubicin; antimetabolites such as methotrexate, 5-fluorouracil (5-FU); folic acid analogs such as denopterin, methotrexate, pteropterin, trimetrexate; pterin analogs such as fludarabine, 6-mercaptopterin, thiomethop-terin, thioguanopterin; pyrimidine analogs such as ancit-abine, azacitidine, 6-azuridine, carmofur, cytarabine, dide-oxyuridine, doxitluridine, enocitabine, floxuridine, 5-FU; androgens such as calusterone, dromostanolong propionate, epitiostanol, mepitiostane, testolactone; anti-adrenalines such as aminoglutethimide, mitotane, trilostane; folic acid supplements such as frolinic acid; aceglatone; aldophosph-amideglycoside; aminolevulinic acid; amsacrine; bestrabu-cil; biasntrene; edatraxate; defofamine; demecolcine; diazi-quone; elfomithine; elliptinium acetate; etoglucid; gallium nitrate; hydroxyurea; lentinan; lonidamine; mitoguazone; mitoxantrone; mopidamol; nitracrine; pintostatin; phe-namet; pirarubicin; podophyllinic acid; 2-ethylhydrazide; procarbazine; PSK®; razoxane; sizofiran; spirogermanium; tenuazonic acid; triaziquone; 2,2′,2″-trichlorrotriethylamine; urethan; vindesine; dacarbazine; mannomustine; mitobroni-tol; dibromodulcitol; pipobroman; gacytosine; arabinoside (“Ara-C”); cyclophosphamide; thiotepa; taxanes such as paclitaxel (TAXOL®, Bristol-Myers Squibb Oncology, Princeton, N.J.) and docetaxel (TAXOTERE®, Rhone-Pou-lenc Rorer, Antony, France); chlorambucil; gemcitabine; 6-thioguanine; mercaptopurine; methotrexate; platinum ana-logs such as cisplatin and carboplatin; vinblastine; platinum; etoposide (VP-16); ifosfamide; mitomycin C; mitoxantrone; vincristine; vinorelbine; navelbine; novantrone; teniposide; daunorubicin; aminopterin; xeloda; ibandronate; CPT-11; topoisomerase inhibitor RFS2000; difluoromethylomithine (DMFO); retinoic acid esperamicins; capecitabine; and pharmaceutically acceptable salt, acid or derivative of any of the above substances. This definition also includes anti-hormonal agents that can modulate or inhibit the effects of hormones on tumors, such as anti-estrogens, including tamoxifen, raloxifene, aromatase inhibitor 4(5)-imidazole, 4-hydroxytamoxifen, trioxifene, keoxifene, LYll 7018, ona-pristone and Fareston; and anti-androgens such as flutamide, nilutamide, bicalutamide, leuprolide and goserelin; and pharmaceutically acceptable salt, acid or derivative of any of the above substances.

The terms “treating” or “treatment” (of, e.g., a disease, disorder, or conditions or associated symptoms, which together or individually may be referred to as “indications”) as used herein include: inhibiting the disease, disorder or condition, i.e., arresting or reducing the development of the disease or its biological processes or progression or clinical symptoms thereof; or relieving the disease, i.e., causing regression of the disease or its biological processes or progression and/or clinical symptoms thereof. “Treatment” as used herein also refers to control, amelioration, or reduction of risks to the subject afflicted with a disease, disorder or condition in which a tumor is involved. The terms “preventing” or “prevention” or “prophylaxis” of a disease, disorder or condition as used herein includes: impeding the development or progression of clinical symptoms of the disease, disorder, or condition in a mammal that may be exposed to or predisposed to the disease, disorder or condition but does not yet experience or display symptoms of the disease, and the like.

As would be evident to those skilled in the art, subjects treated by the methods described herein are generally mammals, including humans and non-human animals (e.g., laboratory animals and companion animals).

The term “composition” as used herein is intended to encompass a product comprising a compound of the disclosure or a pharmaceutically acceptable salt thereof, together with one or more additional specified ingredients in the specified amounts, as well as any product which results, directly or indirectly, from combination of the specified ingredients in the specified amounts. Such term in relation to a pharmaceutical composition, is intended to encompass a product comprising the active ingredient(s), which include a compound of the disclosure or a pharmaceutically acceptable salt thereof, optionally together with one or more additional active ingredients, and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure, or a pharmaceutically acceptable salt thereof, and a pharmaceutically acceptable carrier. By “pharmaceutically acceptable” it is meant the carrier, diluent or excipient must be compatible with the other ingredients of the formulation and not deleterious to the recipient thereof.

As noted above, additional embodiments of the present disclosure are each directed to a method for the treatment a disease, disorder, or condition, or one or more symptoms thereof (“indications”) which method comprises administering to a subject in need of such treatment a therapeutically effective amount of a compound of the disclosure, or a pharmaceutically acceptable salt thereof, or a pharmaceutical composition comprising said compound or salt thereof.

In another embodiment, the present disclosure is directed to a method for the manufacture of a medicament for use in a subject comprising combining a compound of the present disclosure, or a pharmaceutically acceptable salt thereof, with a pharmaceutical carrier or diluent.

One such embodiment provides a method of treating or preventing a cancer selected from breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblastoma, sarcoma, lung cancer (for example, small cell lung cancer and non-small cell lung cancer) colon cancer, rectal cancer, colorectal cancer, leukemia (for example, acute lymphocytic leukemia, acute myeloid leukemia, acute promyelocytic leukemia, chronic myeloid leukemia, chronic lymphocytic leukemia), bone cancer, skin cancer, thyroid cancer, pancreatic cancer, and lymphoma (for example, Hodgkin's lymphoma, non-Hodgkin's lymphoma, or recurrent anaplastic large cell lymphoma) in a subject in need thereof, said method comprising administering to a subject in need of such treatment a therapeutically effective amount of a compound of the disclosure, or a pharmaceutically acceptable salt or solvate thereof, or a pharmaceutical composition comprising said compound, salt or solvate thereof. In one such embodiment, the subject is a human.

Another aspect of the disclosure relates to a method for treating and/or preventing a tumor, comprising administering to a patient in need thereof a therapeutically effective amount of the compound, or a pharmaceutically acceptable salt or solvate thereof, or the pharmaceutical composition comprising the compound according to the present disclosure.

Combinations with additional therapeutic agents are also contemplated in the instant methods. For example, combinations of the compounds of the present disclosure with PPAR-γ (i.e., PPAR-gamma) agonists and PPAR-δ (i.e., PPAR-delta) agonists are useful in the treatment of certain malignancies. PPAR-γ and PPAR-δ are the nuclear peroxisome proliferator-activated receptors γ and δ. PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy) phenoxy)propoxy)-2-ethylchromane-2-carboxylic acid (disclosed in U.S. Ser. Nos 60/235,708 and 60/244,697), or a pharmaceutically acceptable salt thereof.

Another embodiment of the instant disclosure is the use of the compounds of the present disclosure in combination with gene therapy for the treatment of cancer. For an overview of genetic strategies to treating cancer sec Hall et al., (Am. J. Hum. Genet. 61:785-789, 1997) and Kufe et al., (Cancer Medicine, 5th Ed, pp 876-889, B C Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5 (8): 1105-13), and interferon gamma (J. Immunol. 2000; 164:217-222).

The compounds of the disclosure may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR^{9576,} OC144-093, R^{101922,} VX853 and PSC833 (valspodar), or a pharmaceutically acceptable salt thereof.

The compounds of the present disclosure may also be administered with an immunologic-enhancing drug, such as levamisole, isoprinosine and Zadaxin, or a pharmaceutically acceptable salt thereof.

The compounds of the present disclosure may also be useful for treating or preventing cancer in combination with P450 inhibitors including: xenobiotics, quinidine, tyramine, ketoconazole, testosterone, quinine, methyrapone, caffeine, phenelzine, doxorubicin, troleandomycin, cyclobenzaprine, erythromycin, cocaine, furafyline, cimetidine, dextromethorphan, ritonavir, indinavir, amprenavir, diltiazem, terfenadine, verapamil, cortisol, itraconazole, mibefradil, nefazodone and nelfinavir, or a pharmaceutically acceptable salt thereof.

The compounds of the present disclosure may also be useful for treating or preventing cancer in combination with Pgp and/or BCRP inhibitors including: cyclosporin A, PSC833, GF120918, cremophorEL, fumitremorgin C, Ko132, Ko134, Iressa, Imatnib mesylate, EKI-785, C11033, novobiocin, diethylstilbestrol, tamoxifen, resperpine, VX-710, tryprostatin A, flavonoids, ritonavir, saquinavir, nelfinavir, omeprazole, quinidine, verapamil, terfenadine, ketoconazole, nifidepine, FK506, amiodarone, XR^{9576,} indinavir, amprenavir, cortisol, testosterone, LY335979, OC144-093, erythromycin, vincristine, digoxin and talinolol, or a pharmaceutically acceptable salt thereof.

The compounds of the present disclosure may also be useful for treating or preventing cancer, including bone cancer, in combination with bisphosphonates, including but not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

The compounds of the present disclosure may also be useful for treating or preventing breast cancer in combination with aromatase inhibitors. Examples of aromatase inhibitors include but are not limited to: anastrozole, letrozole and exemestane, or a pharmaceutically acceptable salt thereof.

The compounds of the present disclosure may also be useful for treating or preventing cancer in combination with siRNA therapeutics.

The compounds of the present disclosure may also be administered in combination with y-secretase inhibitors and/or inhibitors of NOTCH signaling. Such inhibitors include compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, U.S. Ser. No. 10/957,251, WO 2004/089911, WO 02/081435, WO 02/081433, WO 03/018543, WO 2004/031137, WO 2004/031139, WO 2004/031138, WO 2004/101538, WO 2004/101539 and WO 02/47671 (including LY-450139), or a pharmaceutically acceptable salt thereof.

In one embodiment, specific anticancer agents useful in the present combination therapies include, but are not limited to: pembrolizumab (Keytruda®), abarelix (Plenaxis depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinccard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone injection®); Elliott's B Solution (Elliott's B Solution®); epirubicin (Ellence®); Epoetin alfa (epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®) histrelin acetate (Histrelin implant®); hydroxyurca (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CecBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); Ridaforolimus; sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); Olaparib (Lynparza®) vorinostat (Zolinza®) and zoledronate (Zometa®), or a pharmaceutically acceptable salt thereof.

Thus, the scope of the instant disclosure encompasses the use of the compounds the present disclosure in combination with a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-COA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the therapeutic agents listed above.

Yet another example of the disclosure is a method of treating cancer that comprises administering a therapeutically effective amount of a compound of the present disclosure in combination with paclitaxel or trastuzumab.

The therapeutic combination disclosed herein may be used in combination with one or more other active agents, including but not limited to, other anti-cancer agents that are used in the prevention, treatment, control, amelioration, or reduction of risk of a particular disease or condition (e.g., cell-proliferation disorders). In one embodiment, a compound of the present disclosure is combined with one or more other anti-cancer agents for use in the prevention, treatment, control amelioration, or reduction of risk of a particular disease or condition for which the compounds of the present disclosure are useful. Such other active agents may be administered, by a route and in an amount commonly used therefor, prior to, contemporaneously, or sequentially with a compound of the present disclosure.

The instant disclosure also includes a pharmaceutical composition useful for treating or preventing cancer that comprises a therapeutically effective amount of compounds of the present disclosure and a second compound selected from: an estrogen receptor modulator, an androgen receptor modulator, a retinoid receptor modulator, a cytotoxic/cytostatic agent, an antiproliferative agent, a prenyl-protein transferase inhibitor, an HMG-COA reductase inhibitor, an HIV protease inhibitor, a reverse transcriptase inhibitor, an angiogenesis inhibitor, a PPAR-γ agonist, a PPAR-δ agonist, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the therapeutic agents listed above.

The disclosure further relates to a method of treating cancer in a human patient comprising administration of an and a PD-1 antagonist to the patient. The compound of the disclosure and the PD-1 antagonist may be administered concurrently or sequentially.

In particular embodiments, the PD-1 antagonist is an anti-PD-1 antibody, or antigen binding fragment thereof. In alternative embodiments, the PD-1 antagonist is an anti-PD-L1 antibody, or antigen binding fragment thereof. In some embodiments, the PD-1 antagonist is an anti-PD-1 antibody, independently selected from pembrolizumab, nivolumab, cemiplimab, sintilimab, tislelizumab, atezolizumab (MPDL3280A), camrelizumab and toripalimab. In other embodiments, the PD-L1 antagonist is an anti-PD-L1 antibody independently selected from atezolizumab, durvalumab and avelumab.

In one embodiment, the PD-1 antagonist is pembrolizumab. In particular sub-embodiments, the method comprises administering 200 mg of pembrolizumab to the patient about every three weeks. In other sub-embodiments, the method comprises administering 400 mg of pembrolizumab to the patient about every six weeks.

In further sub-embodiments, the method comprises administering 2 mg/kg of pembrolizumab to the patient about every three weeks. In particular sub-embodiments, the patient is a pediatric patient.

In some embodiments, the PD-1 antagonist is nivolumab. In particular sub-embodiments, the method comprises administering 240 mg of nivolumab to the patient about every two weeks. In other sub-embodiments, the method comprises administering 480 mg of nivolumab to the patient about every four weeks.

In some embodiments, the PD-1 antagonist is cemiplimab. In particular embodiments, the method comprises administering 350 mg of cemiplimab to the patient about every 3 weeks.

In some embodiments, the PD-1 antagonist is atezolizumab. In particular sub-embodiments, the method comprises administering 1200 mg of atezolizumab to the patient about every three weeks.

In some embodiments, the PD-1 antagonist is durvalumab. In particular sub-embodiments, the method comprises administering 10 mg/kg of durvalumab to the patient about every two weeks.

In some embodiments, the PD-1 antagonist is avelumab. In particular sub-embodiments, the method comprises administering 800 mg of avelumab to the patient about every two weeks.

When the compounds of the present disclosure are administered in combination with an anti-human PD-1 antibody (or antigen-binding fragment thereof), the anti-human PD-1 antibody (or antigen-binding fragment thereof) may be administered either simultaneously with, or before or after, the compounds of the present disclosure. Either of the anti-human PD-1 antibody (or antigen-binding fragment thereof), and/or a compound of the present disclosure, or a pharmaceutically acceptable salt thereof, may be administered separately, by the same or different route of administration, or together in the same pharmaceutical composition as the other agent(s). The weight ratio of the anti-human PD-1 antibody (or antigen-binding fragment thereof) to a compound of the present disclosure, may be varied and will depend upon the therapeutically effective dose of each agent. Generally, a therapeutically effective dose of each will be used. Combinations including at least one anti-human PD-1 antibody (or antigen-binding fragment thereof), a compound of the present disclosure, and optionally other active agents will generally include a therapeutically effective dose of each active agent. In such combinations, the anti-human PD-1 antibody (or antigen-binding fragment thereof), the compounds and other active agents may be administered separately or in conjunction. In addition, the administration of one element may be prior to, concurrent with, or subsequent to the administration of other agent(s).

In one embodiment, this disclosure provides an anti-human PD-1 antibody (or antigen-binding fragment thereof), and/or a compound of the disclosure, and at least one other active agent as a combined preparation for simultaneous, separate or sequential use in treating cancer.

The disclosure also provides the use of a compound of the present disclosure, for treating cancer, where the patient has previously (e.g., within 24-hours) been treated with an anti-human PD-1 antibody (or antigen-binding fragment thereof). The disclosure also provides the use of an anti-human PD-1 antibody (or antigen-binding fragment thereof) for treating a cellular proliferative disorder, where the patient has previously (e.g., within 24-hours) been treated with an antibody-linker-payload compound (ADC) a compound of the present disclosure.

The present disclosure further relates to methods of treating cancer, said method comprising administering to a subject in need thereof a combination therapy that comprises (a) a compound of the present disclosure, and (b) an anti-human PD-1 antibody (or antigen-binding fragment thereof); wherein the anti-human PD-1 antibody (or antigen-binding fragment thereof) is administered once every 21 days.

Additionally, the present disclosure relates to methods of treating cancer, said method comprising administering to a subject in need thereof a combination therapy that comprises: (a) a compound of the present disclosure, and (b) an anti-human PD-1 antibody (or antigen-binding fragment thereof. In specific embodiments, the cancer occurs as one or more solid tumors or lymphomas. In further specific embodiments, the cancer is selected from the group consisting of advanced or metastatic solid tumors and lymphomas. In still further specific embodiments, the cancer is selected from the group consisting of malignant melanoma, head and neck squamous cell carcinoma, MSI-H cancer, MMR deficient cancer, non-small cell lung cancer, urothelial carcinoma, gastric or gastroesophageal junction adenocarcinoma, breast adenocarcinoma, and lymphomas. In additional embodiments, the lymphoma is selected from the group consisting of diffuse large B-cell lymphoma, follicular lymphoma, mantle cell lymphoma, small lymphocytic lymphoma, mediastinal large B-cell lymphoma, splenic marginal zone B-cell lymphoma, extranodal marginal zone B-cell lymphoma of mucosa-associated lymphoid tissue (malt), nodal marginal zone B-cell lymphoma, lymphoplasmacytic lymphoma, primary effusion lymphoma, Burkitt lymphoma, anaplastic large cell lymphoma (primary cutaneous type), anaplastic large cell lymphoma (systemic type), peripheral T-cell lymphoma, angioimmunoblastic T-cell lymphoma, adult T-cell lymphoma/leukemia, nasal type extranodal NK/T-cell lymphoma, enteropathy-associated T-cell lymphoma, gamma/delta hepatosplenic T-cell lymphoma, subcutaneous panniculitis-like T-cell lymphoma, mycosis fungoides, and Hodgkin lymphoma. In particular embodiments, the cellular proliferative disorder is a cancer that has metastasized, for example, a liver metastases from colorectal cancer. In additional embodiments, the cellular proliferative disorder is a cancer is classified as stage III cancer or stage IV cancer. In instances of these embodiments, the cancer is not surgically resectable.

In embodiments of the methods disclosed herein, the anti-human PD-1 antibody (or antigen binding fragment thereof) is administered by intravenous infusion or subcutaneous injection.

In one embodiment, the present disclosure provides compositions comprising a compound of the disclosure , a pharmaceutically acceptable carrier, and an anti-human PD-1 antibody (or antigen-binding fragment thereof).

In another embodiment, the present disclosure provides compositions comprising a compound of the disclosure, a pharmaceutically acceptable carrier, and pembrolizumab.

In one embodiment, the present disclosure provides compositions comprising a compound of the disclosure, a pharmaceutically acceptable carrier, and two additional therapeutic agents, one of which is an anti-human PD-1 antibody (or antigen-binding fragment thereof), and the other of which is independently selected from the group consisting of anticancer agents.

A compound of the present disclosure may be employed in conjunction with anti-emetic agents to treat nausea or emesis, including acute, delayed, late-phase, and anticipatory emesis, which may result from the use of a compound of the present disclosure, alone or with radiation therapy. For the prevention or treatment of emesis, a compound of the present disclosure may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide, aprepitant, fosaprepitant, or dronabinol. In another example, conjunctive therapy with an anti-emesis agent selected from a neurokinin-1 receptor antagonist, a 5HT3 receptor antagonist and a corticosteroid is disclosed for the treatment or prevention of emesis that may result upon administration of the compounds of the disclosure.

The compounds of the disclosure may also be administered with an agent useful in the treatment of anemia. Such an anemia treatment agent is, for example, a continuous erythropoiesis receptor activator (such as epoetin alfa).

The compounds of the disclosure may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

The compounds of the disclosure may be useful when co-administered with other treatment modalities, including but not limited to, radiation therapy, surgery, and gene therapy. Accordingly, in one embodiment, the methods of treating cancer described herein, unless stated otherwise, can optionally include the administration of an effective amount of radiation therapy. For radiation therapy, y-radiation is preferred.

The methods of treating cancers described herein can optionally include the administration of an effective amount of radiation (i.e., the methods of treating cancers described herein optionally include the administration of radiation therapy).

The methods of treating cancer described herein include methods of treating cancer that comprise administering a therapeutically effective amount of a compound of Formula III or Formula III′, or Formula III″ in combination with radiation therapy and/or in combination with a second compound selected from: an estrogen receptor modulator, an androgen receptor modulator, a retinoid receptor modulator, a cytotoxic/ytostatic agent, an antiproliferative agent, a prenyl-protein transferase inhibitor, an HMG-COA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase and/or NOTCH inhibitors, agents that interfere with receptor tyrosine kinases (RTKs), an agent that interferes with a cell cycle checkpoint, and any of the additional therapeutic agents listed herein.

Additional embodiments of the disclosure include the pharmaceutical compositions, combinations, uses and methods set forth in above, wherein it is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

### Kits

In one aspect, provided is a kit comprising a therapeutically effective amount of a compound of the present disclosure or a pharmaceutically acceptable salt, solvate or ester of said compound and a pharmaceutically acceptable carrier, vehicle or diluent.

In another aspect provided is a kit comprising an amount of a compound of the present disclosure, and an amount of at least one additional therapeutic agent listed above, wherein the amounts of the two or more active ingredients result in a desired therapeutic effect. In one embodiment, the compound of the present disclosure, and the one or more additional therapeutic agents are provided in the same container. In one embodiment, the compound of the present disclosure, and the one or more additional therapeutic agents are provided in separate containers.

The present disclosure includes within its scope prodrugs of the compounds of this disclosure. In general, such prodrugs will be functional derivatives of the compounds of this disclosure which are readily convertible in vivo into the required compound. Thus, in the methods of treatment of the present disclosure, the terms “administration of” or “administering a” compound shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this disclosure into the biological milieu.

The compounds described herein, or pharmaceutically acceptable salts and/or solvates thereof, may be administered singly, in combination with other compounds of the disclosure, and/or in cocktails combined with other therapeutic agents. The choice of therapeutic agents that can be co-administered with the compounds of the disclosure will depend, in part, on the condition being treated.

The compounds of the present disclosure may be administered by oral, parenteral (e.g., intramuscular, intraperitoneal, intravenous, intracerebroventricular (ICV), intracisternal injection or infusion, subcutaneous injection, or implant), by inhalation spray, nasal, vaginal, rectal, sublingual, buccal or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals the compounds of the disclosure are effective for use in humans.

The pharmaceutical compositions for the administration of the compounds of this disclosure may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, solutions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated, or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256, 108; 4, 166,452; and 4,265,874 to form osmotic therapeutic tablets for control release. Oral tablets may also be formulated for immediate release, such as fast melt tablets or wafers, rapid dissolve tablets or fast dissolve films.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecacthyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or acetyl alcohol. Sweetening agents such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compounds of the present disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions and the like, containing the compounds of the present disclosure are employed. Similarly, transdermal patches may also be used for topical administration.

The pharmaceutical composition and method of the present disclosure may further comprise other therapeutically active compounds as noted herein which are usually applied in the treatment of the above-mentioned pathological conditions.

In the treatment, prevention, control, amelioration, or reduction of risk of the conditions disclosed herein an appropriate dosage level of the compounds of this disclosure will generally be about 0.01 to 500 mg per kg patient body weight per day which can be administered in single or multiple doses. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day or may be administered once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Methods for preparing the compounds of this disclosure are illustrated in the following Schemes and Examples. Starting materials are made according to procedures known in the art or as illustrated herein.

### PREPARATIVE EXAMPLES

The compounds of the present disclosure can be prepared according to the following schemes and specific examples, or modifications thereof, using readily available starting materials, reagents and conventional synthesis procedures. It is also possible to make use of variants which are themselves known to those of ordinary skill in this art but are not mentioned in detail. The general procedures for making the compounds claimed in this disclosure can be readily understood by one skilled in the art from viewing the following schemes and descriptions. Abbreviations used in the experimentals may include, but are not limited to the following:

| | |
|---|---|
| ACN | Acetonitrile |
| Aq | Aqueous |
| DAR | Drug to antibody ratio |
| DCM | Dichloromethane |
| DIPEA | N,N-Diisopropylethylamine |
| DMF | Dimethylformamide |
| DMSO | Dimethyl sulfoxide |
| ESI | Electron-spray ionization |
| EtOAc | Ethyl acetate |
| FBS | Fetal bovine serum |
| Et₃N | Triethylamine |
| H | Hours |
| HATU | (1-[Bis(dimethylamino)methylene]-1H-1,2,3-triazolo[4,5- |
| | b]pyridinium 3-oxide hexafluorophosphate |
| ¹H-NMR | Proton nuclear magnetic resonance |
| HCl | Hydrochloric acid |
| HOBt | Hydroxybenzotriazole |
| HPLC | High performance liquid chromatography |
| IPA | Isopropyl alcohol |
| LCMS | Liquid chromatography-mass spectrometry |
| m-CPBA | meta-Chloroperoxybenzoic acid |
| MeCN | Acetonitrile |
| MeOH | Methanol |
| MMAE | Monomethyl auristatin E |
| MS | Mass spectrometry |
| MTBE | Methyl Tert-butyl ether |
| m/z | Mass to charge ratio |
| NMP | N-Methyl-2-pyrrolidone |
| PBS | Phosphate buffered saline |
| PyBOP | Benzotriazol-1-yl-oxytripyrrolidinophosphonium |
| | hexafluorophosphate |
| RT | Room temperature |
| TFA | Trifluoroacetic acid |
| THF | Tetrahydrofuran |
| TLC | Thin Layer Chromatography |
| t_{R} | Retention time |
| TEA | Tricthylamine |
| Celite | Trademark for diatomaceous earth |
| DIEA | Diisopropyl ethyl amine |

### Preparation of Intermediate Compounds

The following Preparative Examples 1-3 describe the synthesis of intermediate compounds useful for making the illustrative compounds of the present disclosure.

### Preparative Example 1

Preparation of Intermediate tert-butyl L-alanyl-L-alaninate (iii)

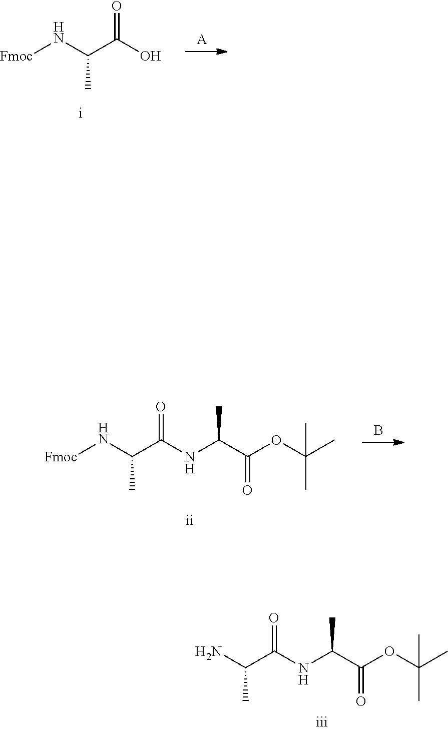

### Step A—Synthesis of Compound ii

To (((9H-fluoren-9-yl)methoxy)carbonyl)-L-alanine (i) (140 g, 450 mmol) dissolved with DCM (4500 mL) was added PyBOP (281 g, 540 mmol, 1.2 eq) at room temperature. The reaction mixture stirred for 20 minutes. tert-Butyl L-alaninate HCl (85.6 g, 471 mmol) was added, and the solution was cooled to 0° C. DIEA (175 g, 1.35 mol) was then added dropwise into the reaction over 1 hour, and it was then stirred overnight at 20° C. The reaction mixture was concentrated in vacuo, diluted with ethyl acetate and washed with aqueous sodium carbonate (1 M), potassium bisulfate (1 M), water, and brine. The organic phase was then concentrated in vacuo and evaporated to 25% of the original solvent volume. MTBE (1400 mL) was added dropwise, and the mixture was stirred for 5 hours. The suspension was filtered, and the solids were washed with MTBE to provide tert-butyl (((9H-fluoren-9-yl)methoxy)carbonyl)-L-alanyl-L-alaninate (ii). LCMS: (ESI, m/z): [M+H]⁺=439.2.

### Step B—Synthesis of Compound iii

To tert-butyl (((9H-fluoren-9-yl)methoxy)carbonyl)-L-alanyl-L-alaninate (ii) (172 g, 392 mmol) dissolved in DCM (2000 mL) was added TEA (2000 mL). The reaction mixture was warmed to 40° C. and stirred for 16 hours. The reaction mixture was then concentrated in vacuo and purified using silica gel chromatography (DCM: 4 M NH₃ in MeOH 5:1) to provide tert-butyl L-alanyl-L-alaninate (iii). LCMS: (ESI, m/z): [M+H]⁺=217.1.

### Preparative Example 2

Preparation of Intermediate Compound 3-(5-cyano-6-methylsulfonyl)nicotinamido)propanoic acid (x)

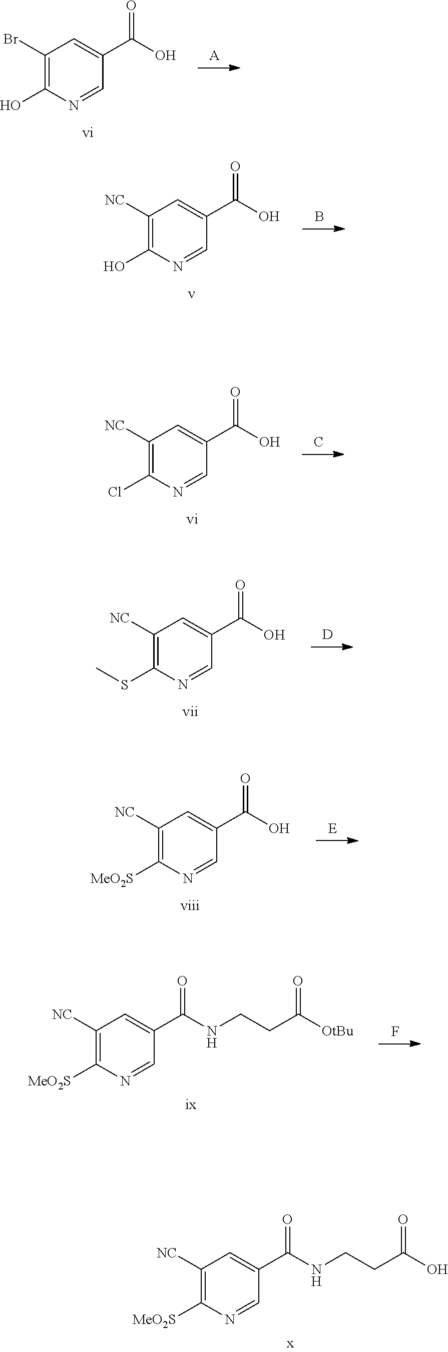

### Step A—Synthesis of Compound v

A solution of 5-bromo-6-hydroxypyridine-3-carboxylic acid (iv, 42.8 g, 196 mmol) and cuprous cyanide (35.2 g, 393 mmol) in NMP (430 mL) was stirred for 2 hours at 165° C. under a N₂ atmosphere. The mixture was concentrated in vacuo, and the crude product was purified using reverse phase flash chromatography (AQ C18 silica gel, ACN in water 0% to 20% gradient (with 0.5% NH₃·H₂O as modifier)) to provide crude product. The resulting mixture was filtered, and the filter cake was washed with H₂O. The filtrate was concentrated in vacuo to provide 5-cyano-6-hydroxypyridine-3-carboxylic acid (v). LCMS: (ESI, m/z): [M−H]⁻=163.

### Step B—Synthesis of Compound vi

A solution of 5-cyano-6-hydroxypyridine-3-carboxylic acid (v) (25.7 g, 157 mmol) and phosphorus oxychloride (130 mL) was stirred for 2 hours at 110° C. The reaction was monitored by LCMS and then concentrated in vacuo. Water and EtOAc cooled to 10° C. were added, and the solid was filtered out. The mixture was extracted with ethyl acetate, dried and concentrated in vacuo. The residue was purified using reverse phase flash chromatography (column AQ silica gel, 0% to 15% acetonitrile/sodium bicarbonate (aq.)) The pH value of the aqueous layer was adjusted to 2-3 with 1 M HCl and then the aqueous layer was extracted with ethyl acetate (3×), dried, and concentrated in vacuo to provide 6-chloro-5-cyanopyridine-3-carboxylic acid (vi). LCMS: (ESI, m/z): [M−H]⁻=181.

### Step C Synthesis of Compound vii

Into a 500 mL three-necked bottle under a nitrogen atmosphere was added dimethylformamide (115 mL) and 6-chloro-5-cyanopyridine-3-carboxylic acid (vi) (7.7 g, 42 mmol) at 25° C. To the above mixture was added (methylsulfanyl)sodium (7.39 g, 105 mmol) in portions at 0° C. The resulting mixture was stirred for 8 hours at 25° C. The reaction was monitored by LCMS. This solution was slowly transferred to H₂O (1200 mL), then extracted with ethyl acetate (1×700 mL). The pH value of the aqueous layer was adjusted to 2-3 with 1 M HCl. The solid was collected by filtration to provide 5-cyano-6-(methylsulfanyl)pyridine-3-carboxylic acid (vii). LCMS: (ESI, m/z): [M−H]⁻=193.

Step D Synthesis of Compound viii

A solution of 5-cyano-6-(methylsulfanyl)pyridine-3-carboxylic acid (vii) (7.4 g, 38 mmol) in DCM (185 mL) was treated with m-CPBA (26.3 g, 152 mmol) for 24 hours at 45° C. under nitrogen atmosphere. The reaction was monitored by LCMS and then quenched with saturated sodium bisulfite at 0° C. and concentrated in vacuo. To the residue was added 2-methyltetrahydrofuran, and then the mixture was filtered and concentrated in vacuo to provide the crude product. The residue was purified using silica gel column chromatography eluting with DCM/MeOH to afford 5-cyano-6-(methylsulfonyl)nicotinic acid (viii). LCMS: (ESI, m/z): [M+H]⁺=227.05.

### Step E Synthesis of Compound ix

5-Cyano-6-(methylsulfonyl)nicotinic acid (viii) (0.57 g, 2.5 mmol) and HATU (1.0 g, 2.7 mmol) were dissolved in 10 mL DMF and stirred for 30 minutes at 25° C. Then tert-butyl 3-aminopropanoate (0.42 g, 2.8 mmol) was added, and the mixture was cooled to 10° C. DIPEA (0.873 mL, 5.00 mmol) was added dropwise into the reaction mixture at 10° C. The reaction mixture was stirred at 25° C. for 2 hours and then diluted with water and extracted with EtOAc (3×). The organic phase was concentrated in vacuo, and the residue was purified using silica gel column chromatography eluting with 2:1 to 1:1 hexanes:EtOAc to provide tert-butyl 3-(5-cyano-6-(methylsulfonyl)nicotinamido)propanoate (ix). ¹H NMR (500 MHZ, CD₃OD) δ 8.49 (d, J=2.0 Hz, 1H), 8.07 (d, J=2.0 Hz, 1H), 4.12 (s, 3H), 2.92 (t, J=6.9 Hz, 2H), 1.88 (t, J=6.9 Hz, 2H), 0.74 (s, 9H).

### Step F—Synthesis of Compound x

tert-Butyl 3-(5-cyano-6-(methylsulfonyl)nicotinamido)propanoate (ix) (0.10 g, 0.28 mmol) was dissolved in 10 mL 1,4-dioxane. Then 4 M HCl in 1,4-dioxane (10 mL) was added into the mixture. The resulting mixture was stirred at 25° C. for 16 hours, and the reaction mixture was filtered. The solid was washed with n-heptane and dried under nitrogen for 5 hours to provide 3-(5-cyano-6-(methylsulfonyl)nicotinamido)propanoic acid (x). ¹H NMR (500 MHZ, CD₃OD) δ 9.22 (d, J=1.9 Hz, 1H), 8.80 (d, J=1.9 Hz, 1H), 3.66 (t, J=5.8 Hz, 2H), 3.44 (s, 3H), 2.67 (t, J=6.8 Hz, 2H).

### Preparative Example 3

Preparation of Intermediate Compound 4-((S)-2-((S)-2-aminopropanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (xiv)

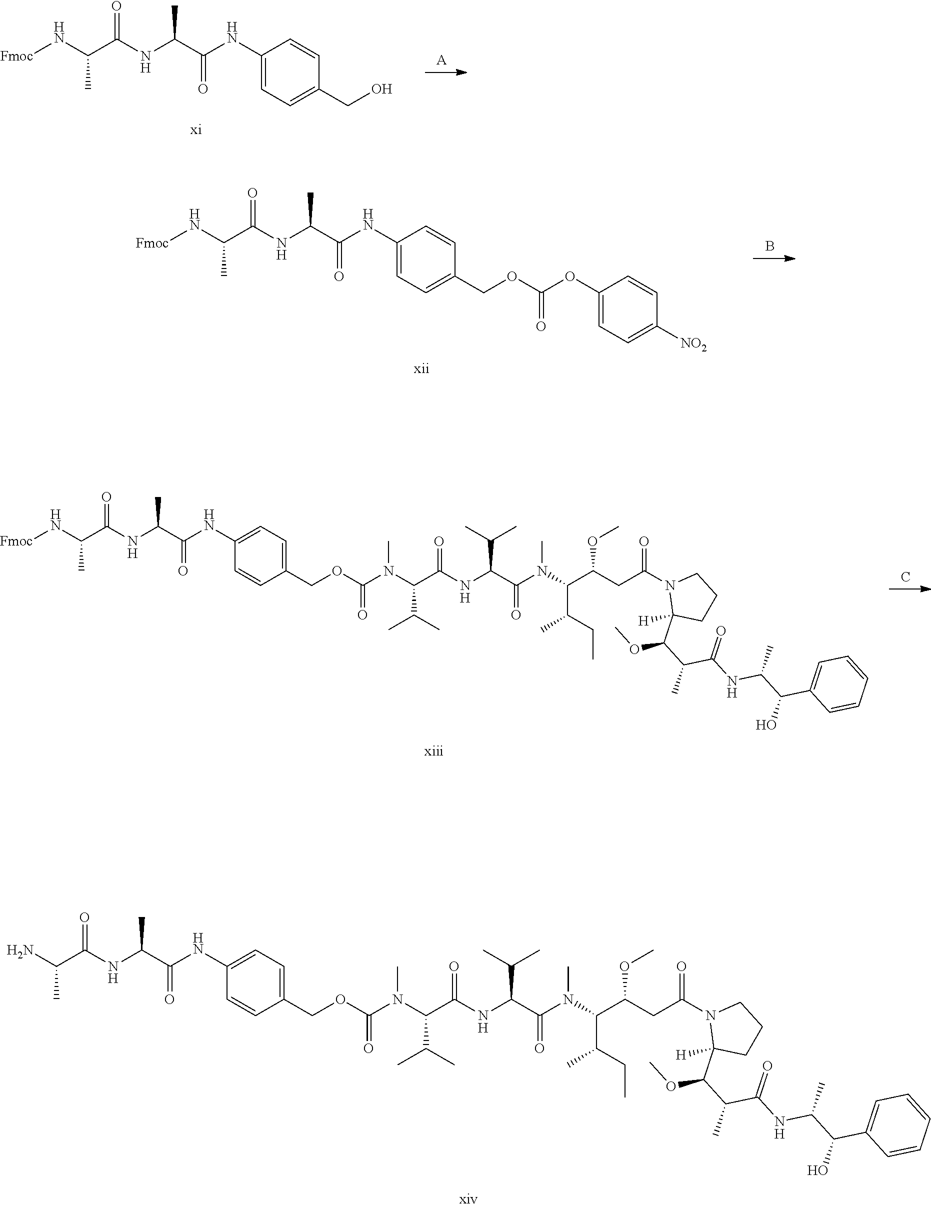

### Step A—Synthesis of Compound xii

A solution of (9H-fluoren-9-yl)methyl ((S)-1-(((S)-1-((4-(hydroxymethyl)phenyl)amino)-1-oxopropan-2-yl)amino)-1-oxopropan-2-yl)carbamate (xi) (13 g, 27 mmol, 1 eq) in DMF (130 mL,) was added into a 500 mL 4-necked round-bottom flask purged and maintained with an inert atmosphere of nitrogen. Then 2 mol % DIEA (0.534 mmol) and bis(4-nitrophenyl) carbonate (16.3 g, 53.6 mmol, 2 eq) were added into the reaction mixture at 20° C. The reaction mixture was warmed to 45° C. and stirred for 16 hours. The reaction mixture was cooled to room temperature and purified using reverse phase flash column chromatography (30% to 60%, MeCN/water with 0.05% TFA as modifier) to provide (9H-fluoren-9-yl)methyl ((S)-1-(((S)-1-((4-((((4-nitrophenoxy)carbonyl)oxy)methyl)phenyl)amino)-1-oxopropan-2-yl)amino)-1-oxopropan-2-yl)carbamate (xii). LCMS: (ESI, m/z): [M+H]⁺=653.2.

Step B Synthesis of Compound xiii

A solution of (9H-fluoren-9-yl)methyl ((S)-1-(((S)-1-((4-((((4-nitrophenoxy)carbonyl)oxy)methyl)phenyl)amino)-1-oxopropan-2-yl)amino)-1-oxopropan-2-yl)carbamate (xii) (8.30 g, 12.7 mmol) in DMF (83 mL) was added into a 250 mL 4-necked round-bottom flask purged and maintained with an inert atmosphere of nitrogen. Then HOBt (340 mg, 2.5 mmol) was added, and the reaction mixture was stirred for 10 minutes at room temperature. MMAE (9 g, 12.7 mmol) was then added to the reaction mixture, and the resulting mixture was stirred for 16 hours at room temperature. The reaction mixture was purified using reverse phase flash column chromatography (20% to 50% MeCN/water with 0.05% TFA as modifier) to provide 4-((S)-2-((S)-2-((((9H-fluoren-9-yl)methoxy)carbonyl)amino)propanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (xiii). LCMS: (ESI, m/z): [M+H]⁺=1231.7.

### Step C Synthesis of Compound xiv

4-((S)-2-((S)-2-((((9H-Fluoren-9-yl)methoxy)carbonyl)amino)propanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (xiii) (0.092 g, 0.075 mmol) was dissolved in DCM (0.5 mL) and TEA (0.5 mL) was added into the reaction mixture. The reaction mixture was stirred for 16 hours at 40° C. The reaction mixture which contained 4-((S)-2-((S)-2-aminopropanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (xiv) was used directly without further purification or concentration. LC-MS: (ESI, m/z): [M+H]⁺=1009.8.

### Preparation of Illustrative Compounds

The following Examples 1-3 describe the synthesis of illustrative compounds of the present disclosure.

### Example 1-4-((S)-2-((S)-2-(3-(2,5-dioxo-2,5-dihydro-1H-pyrrol-1-yl)propanamido)propanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (1)

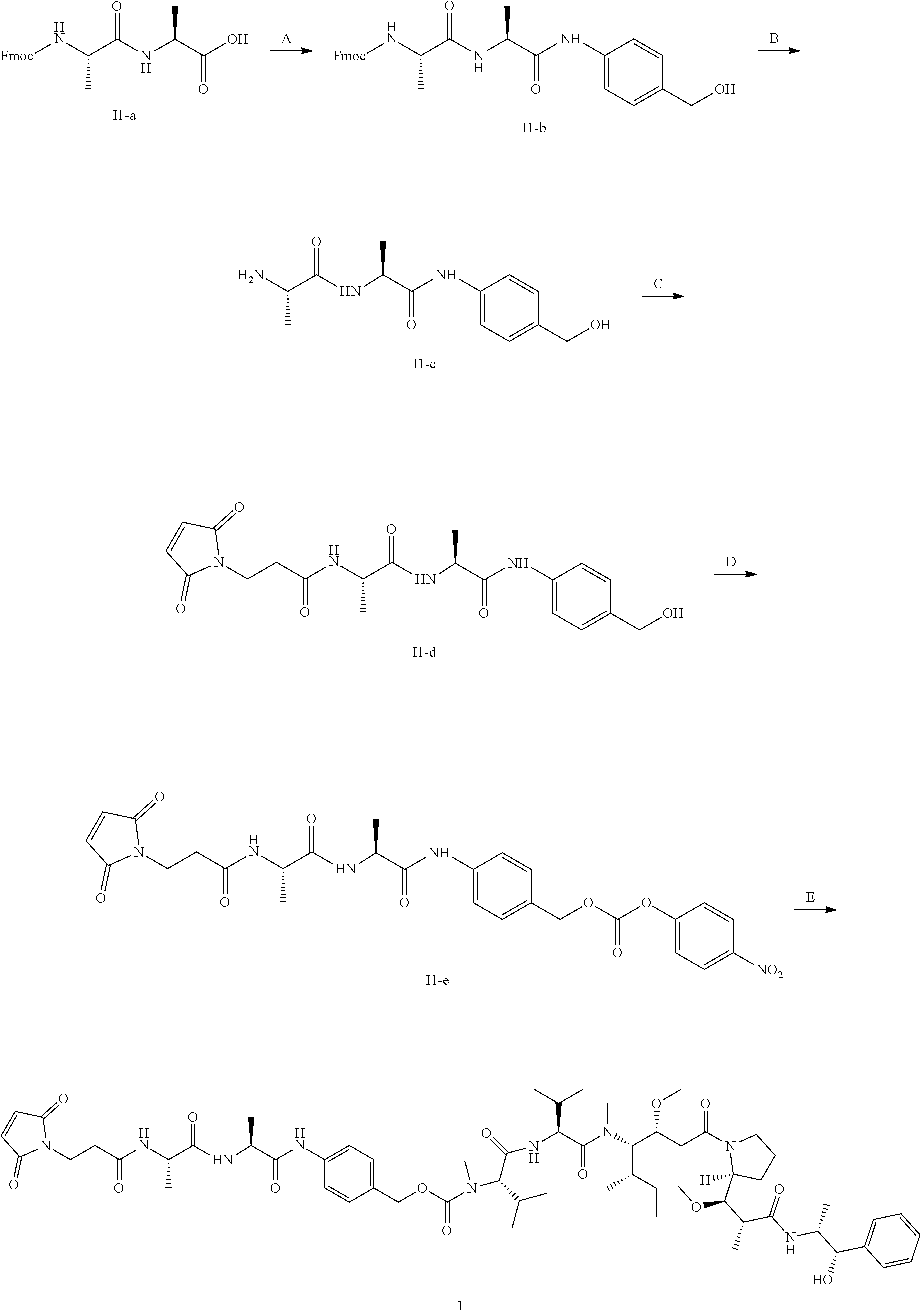

### Step A—Synthesis of Compound I1-b

P-aminobenzylalcohol (154 g, 125 mmol) was added to a stirred mixture of ethyl 2-ethoxy-2H-quinoline-1-carboxylate (103 g, 418 mmol) and (2S)-2-[(2S)-2-{[(9H-fluoren-9-ylmethoxy)carbonyl]amino}propanamido]propanoic acid I1-a (100 g, 261 mmol) in DCM:MeOH (2:1) (3000 mL) at 0° C. The reaction mixture was stirred at room temperature for 18 hours and monitored by LCMS. The solvent was evaporated in vacuo, and the residue was diluted with TBME (2000 mL) and stirred for 30 minutes. The solids were collected by filtration, washed with TBME (1000 mL) and dried in vacuo to provide 9H-fluoren-9-ylmethyl N-[(1S)-1-{[(1S)-1-{[4-(hydroxymethyl)phenyl]carbamoyl}ethyl]-carbamoyl}ethyl]carbamate (I1-b). LCMS: (ES, m/z): [M+H]⁺=488.

### Step B—Synthesis of Compound I1-c

Diethylamine (756 g, 10.3 mol) was added to a stirred mixture of 9H-fluoren-9-ylmethyl N-[(1S)-1-{[(1S)-1-{[4-(hydroxymethyl)phenyl]carbamoyl}ethyl]-carbamoyl}ethyl]carbamate (I1-b) (140 g, 287 mmol) in DMF (1.4 L) at room temperature. The mixture was stirred at room temperature for 18 hours. The reaction was monitored by LCMS. The solvent was evaporated, and the residue was diluted with EtOAc (500 mL) and stirred for 30 minutes. The solids were collected by filtration, washed with EtOAc (300 mL), and dried in vacuo to provide (2S)-2-[(2S)-2-aminopropanamido]-N-[4-(hydroxymethyl)phenyl]propanamide (I1-c). LCMS: (ES, m/z): [M+H]⁺=266.

### Step C Synthesis of Compound I1-d

A mixture of 2,5-dioxopyrrolidin-1-yl 3-(2,5-dioxopyrrol-1-yl)propanoate (55.2 g, 207 mmol), I1-c (55.0 g, 207 mmol) and DIPEA (40.2 g, 311 mmol) in DMF (550 mL) at room temperature was stirred for 16 hours. The reaction mixture was added to H₂O (600 mL) with stirring. The solids were collected by filtration, washed with water (500 mL), and dried in vacuo to provide (2S)-2-[3-(2,5-dioxopyrrol-1-yl)propanamido]-N-[(1S)-1-{[4-(hydroxymethyl)phenyl]carbamoyl}ethyl]propanamide (I1-d) that was used directly in the next step.

### Step D Synthesis of Compound I1-e

DIPEA (30.7 g, 237 mmol) was added to a stirred mixture of bis(4-nitrophenyl) carbonate (60.3 g, 198 mmol) and (2S)-2-[3-(2,5-dioxopyrrol-1-yl)propanamido]-N-[(1S)-1-{[4-(hydroxymethyl)phenyl]carbamoyl}ethyl]propanamide (I1-d) (55 g, 32 mmol) in DMF (550 mL) at room temperature. The mixture was stirred for 16 hours and monitored by LCMS. The reaction solution was added to H₂O (400 mL) with stirring. The mixture was filtered, and the filter cake was washed with water (1×100 mL). The solids were collected by filtration and purified using reverse phase chromatography (Dynamic axial chromatographic column C-18, eluting with 15% to 65% ACN/Water (with 0.1% ammonium acetate (NH₄OAc) as modifier)). The resulting mixture was concentrated in vacuo, and the solids were dried in vacuo at 45° C. to provide {4-[(2S)-2-[(2S)-2-[3-(2,5-dioxopyrrol-1-yl)propanamido]propanamido]propanamido]phenyl}methyl-4-nitrophenyl carbonate (I1-e). LC-MS: (ES, m/z): [M+H]⁺=582.

### Step E Synthesis of 1

To a 60 mL round-bottomed flask under N₂ was added {4-[(2S)-2-[(2S)-2-[3-(2,5-dioxopyrrol-1-yl)propanamido]propanamido]propanamido]phenyl}methyl-4-nitrophenyl carbonate (I1-e) (3.00 g, 5.15 mmol) and DMF (45 mL), followed by HOBt (140 mg, 1.03 mmol). The reaction mixture was stirred at 25° C. for 10 minutes, after which time MMAE (4.00 g. 5.57 mmol) was added at 25° C. The reaction mixture was stirred at 25° C. for 16 hours. The reaction was then purified using reverse phase column chromatography (AQ C18 30% to 60% MeCN/water with 0.1% ammonium acetate as modifier). The MeCN was concentrated in vacuo, the remaining aqueous mixture extracted with ethyl acetate (300 ml×3), and the combined organics were concentrated in vacuo. Four batches were run in parallel, combined, and dissolved in 200 mL of MeCN. 400 mL water was added, and the solution was frozen and then lyopholized to afford 4-((S)-2-((S)-2-(3-(2,5-dioxo-2,5-dihydro-1H-pyrrol-1-yl)propanamido)propanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (1). LC-MS: (ES, m/z): [M+H]⁺=1160. ¹H NMR (400 MHZ, CD₃OD) δ 7.53 (br d, J=8.19 Hz, 2H), 7.03-7.32 (m, 7H), 6.67 (s, 2H), 5.21-5.29 (m, 2H), 4.90-5.14 (m, 2H), 4.28-4.66 (m, 4H), 4.04-4.22 (m, 4H), 3.52-3.81 (m, 4H), 3.24-3.46 (m, 6H), 3.17 (s, 2H), 2.97-3.09 (m, 2H), 2.73-2.90 (m, 4H), 2.28-2.47 (m, 4H), 1.43-2.20 (m, 8H), 1.16-1.38 (m, 10H), 1.00-1.12 (m, 6H), 0.61-0.94 (m, 18H) Not all exchangeable protons are reported.

### Example 2: 4-((S)-2-((S)-2-(3-(5-cyano-6-(methylsulfonyl)picolinamido)propanamido)propanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (2)

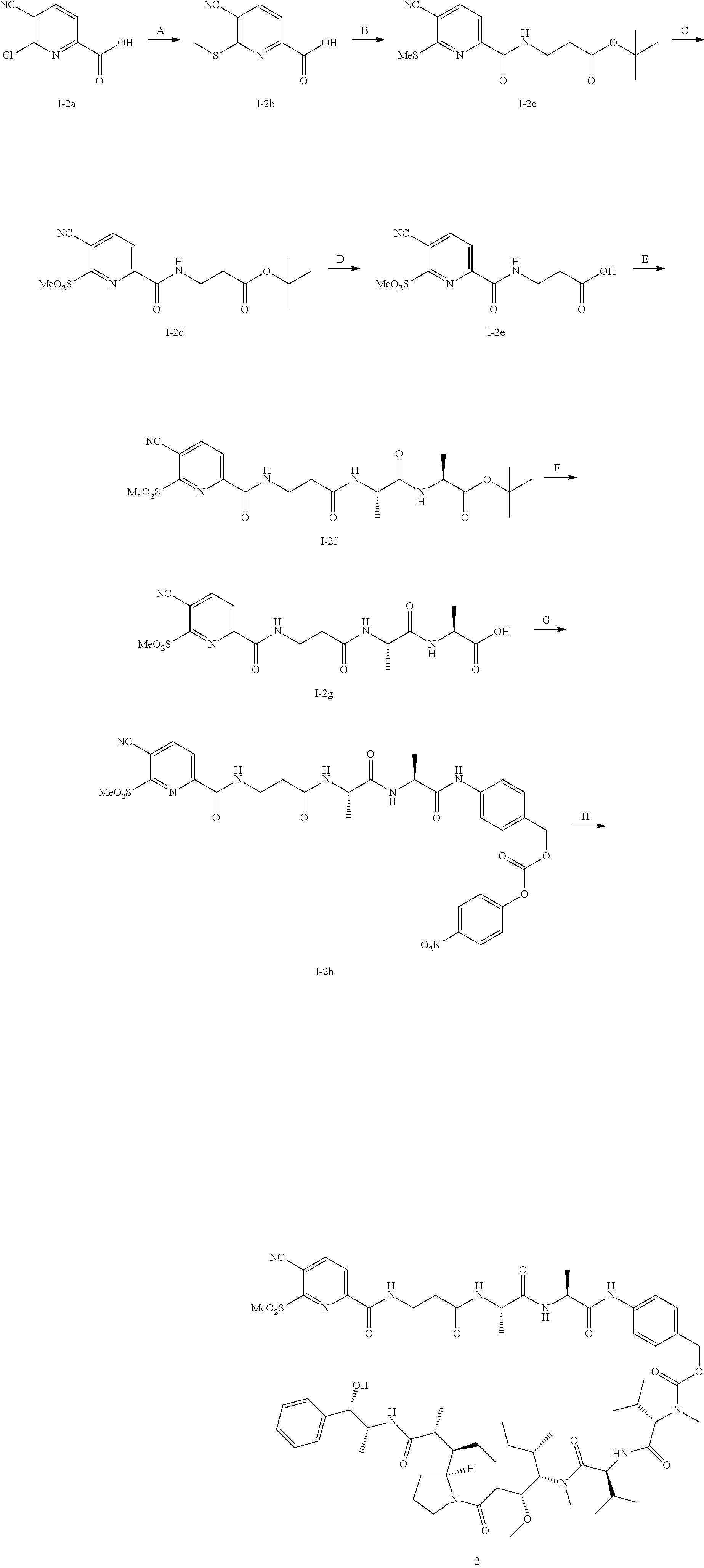

### Step A—Synthesis of Compound I-2b

6-Chloro-5-cyanopicolinic acid (I-2a) (80 g, 0.44 mol) was dissolved in DMF (5000 mL), and sodium methane thiolate (77 g, 1.1 mol) was added into the mixture in batches. The reaction mixture was stirred at 25° C. for 16 hours. The reaction mixture was then diluted with ethyl acetate and added to water. The mixture was extracted with ethyl acetate and the aqueous phase was adjusted to pH 5 with 10% citric acid. The mixture was extracted with ethyl acetate 3 times and the combined organics were concentrated in vacuo to provide 5-cyano-6-(methylthio)picolinic acid (I-2b). LC-MS: (ES, m/z): [M+H]⁺=195.

### Step B—Synthesis of Compound I-2c

To 5-cyano-6-(methylthio)picolinic acid (I-2b) (66 g, 0.34 mol) dissolved in THF (3000 mL) was added HATU (155 g, 0.41 mol). The resulting mixture was stirred for 30 minutes at 25° C. tert-Butyl 3-aminopropanoate HCl salt (67.7 g, 0.37 mol) was added into the mixture and then it was cooled to 10° C. DIEA (175 g, 1.35 mol) was added dropwise over 1 hour at 10° C. The reaction mixture was stirred at 25° C. for 16 hours, concentrated in vacuo and diluted with ethyl acetate. The resulting solution was washed with water 3 times and the combined organics were concentrated in vacuo. The residue was purified using silica gel column chromatography (ethyl acetate:petroleum ether 1:2) to provide tert-butyl 3-(5-cyano-6-(methylthio)picolinamido)propanoate (I-2c). LC-MS: (ES, m/z): [M+Na]⁺=344

### Step C—Synthesis of Compound I-2d

To tert-Butyl 3-(5-cyano-6-(methylthio)picolinamido)propanoate (I-2c). (96 g, 0.30 mol) was dissolved in DCM (1500 mL) and a solution of m-CPBA (206 g, 1.19 mol) in DCM (1500 mL) was added dropwise at 0° C. The reaction mixture was stirred at room temperature for 16 hours. The resulting reaction mixture was diluted with DCM and poured into ice water. The organic phase was washed with 10% sodium bicarbonate in water 4 times and the combined organics were dried and concentrated in vacuo to provide tert-butyl 3-(5-cyano-6-(methylsulfonyl)picolinamido)propanoate (I-2d). LC-MS: (ES, m/z): [M+Na]⁺=376

### Step D—Synthesis of Compound I-2e

To tert-butyl 3-(5-cyano-6-(methylsulfonyl)picolinamido)propanoate (I-2d) (90 g, 0.25 mol) dissolved in 1,4-dioxane (1000 mL) was added 4 M HCl in 1,4-dioxane (2500 mL). The resulting mixture was stirred at 25° C. for 16 hours and then filtered. The solid was washed with n-heptane and dried under nitrogen to provide 3-(5-cyano-6-(methylsulfonyl)picolinamido)propanoic acid (I-2e). LC-MS: (ES, m/z): [M+Na]⁺=320

### Step E—Synthesis of Compound I-2f

To tert-Butyl L-alanyl-L-alaninate (iii) (50 g, 0.23 mol) was dissolved in THF (2300 mL). To this mixture was added 3-(5-cyano-6-(methylsulfonyl)picolinamido)propanoic acid (I-2e) (69 g, 0.23 mol) and HATU (106 g, 0.28 mol). The reaction mixture was stirred for 30 minutes and then cooled to 10° C. DIEA (90 g) was then added dropwise over 30 minutes. The reaction mixture was stirred overnight at 20° C. The reaction mixture was then concentrated in vacuo, and the resulting residue was diluted with ethyl acetate. The combined organics were washed with water 3 times, dried, and concentrated in vacuo. The resulting residue was purified using silica gel chromatography with DCM: MeOH (2:1) to provide tert-butyl (3-(5-cyano-6-(methylsulfonyl)picolinamido)propanoyl)-L-alanyl-L-alaninate (I-2f). LC-MS: (ES, m/z): [M+Na]⁺=518

### Step F Synthesis of Compound I-2g

4 M HCl in 1,4-dioxane (1800 mL) was added to tert-butyl (3-(5-cyano-6-(methylsulfonyl)picolinamido)propanoyl)-L-alanyl-L-alaninate (I-2f) (90 g, 0.18 mol) dissolved in MeCN (2000 mL). The reaction mixture was stirred at 25° C. for 16 hours, then concentrated in vacuo. The resulting solid was washed with MTBE and filtered. The solid was air-dried overnight to provide (3-(5-cyano-6-(methylsulfonyl)picolinamido)propanoyl)-L-alanyl-L-alanine (I-2g). LC-MS: (ES, m/z): [M+Na]⁺=462

### Step G—Synthesis of Compound I-2h

Into a 500 mL 4-necked round-bottom flask was purged and maintained with an inert atmosphere of nitrogen, was added (3-(5-cyano-6-(methylsulfonyl)picolinamido)propanoyl)-L-alanyl-L-alanine (I-2g) (30 g, 0.65 mol), DMF (300 mL), and HATU (31.2 g, 0.780 mol). The reaction mixture was stirred for 30 minutes at room temperature. Then (4-aminophenyl)methanol (9.0 g, 0.068 mmol) was added at 20° C., and the reaction mixture was cooled to 10° C. DIEA (90 g, 0.19 mmol) was added dropwise into the reaction mixture over 30 minutes at 10° C., and the resulting mixture was stirred at 25° C. for 5 hours. Then bis(4-nitrophenyl) carbonate (42 g, 0.13 mmol) was added into reaction mixture at 25° C., and stirred at 25° C. for 1 hour. The reaction mixture was purified using C-18 flash column chromatography (30% to 60% ACN/water (with 0.05% TFA as modifier)) to provide 4-((S)-2-((S)-2-(3-(5-cyano-6-(methylsulfonyl)picolinamido)propanamido)propanamido)propanamido)benzyl (4-nitrophenyl) carbonate (I-2h). LC-MS: (ES, m/z): [M+Na]⁺=732

### Step H—Synthesis of Compound 2

To 4-((S)-2-((S)-2-(3-(5-cyano-6-(methylsulfonyl)picolinamido)propanamido)propanamido)propanamido)benzyl (4-nitrophenyl) carbonate (I-2h) (5.6 g, 7.9 mmol) and 1H-benzo[d][1,2,3] triazol-1-ol (0.213 g, 1.58 mmol) was added DMF (56.0 mL). MMAE (5.67 g, 7.89 mmol) was added to the reaction at 20° C. and the reaction mixture was stirred at 40° C. 16 hours. The resulting mixture was purified using Prep-HPLC (10-95% MeCN/water, 0.05% TFA) to provide 4-((S)-2-((S)-2-(3-(5-cyano-6-(methylsulfonyl)picolinamido)propanamido)propanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (2). ¹H NMR (400 MHZ, DMSO-d6) δ 9.86 (s, 1H), 9.14 (t, J=6.0 Hz, 1H), 8.79 (d, J=8.1 Hz, 1H), 8.38 (d, J=8.1 Hz, 1H), 8.33-7.97 (m, 3H), 7.96-7.51 (m, 3H), 7.41-6.87 (m, 7H), 6.09 (s, 1H), 5.37 (dd, J=26.2, 5.0 Hz, 1H), 5.04 (tt, J=25.6, 12.6 Hz, 2H), 4.69 (d, J=44.2 Hz, 1H), 4.59-4.16 (m, 5H), 4.15-3.90 (m, 2H), 3.80-3.43 (m, 8H), 3.36-2.95 (m, 9H), 2.94-2.58 (m, 2H), 2.50-2.20 (m, 3H), 2.19-1.79 (m, 3H), 1.65-1.37 (m, 3H), 1.32-1.18 (m, 9H), 1.06-0.96 (m, 7H), 0.89-0.59 (m, 20H).

### Example 3: 4-((S)-2-((S)-2-(3-(5-cyano-6-(methylsulfonyl)nicotinamido)propanamido)propanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (3)

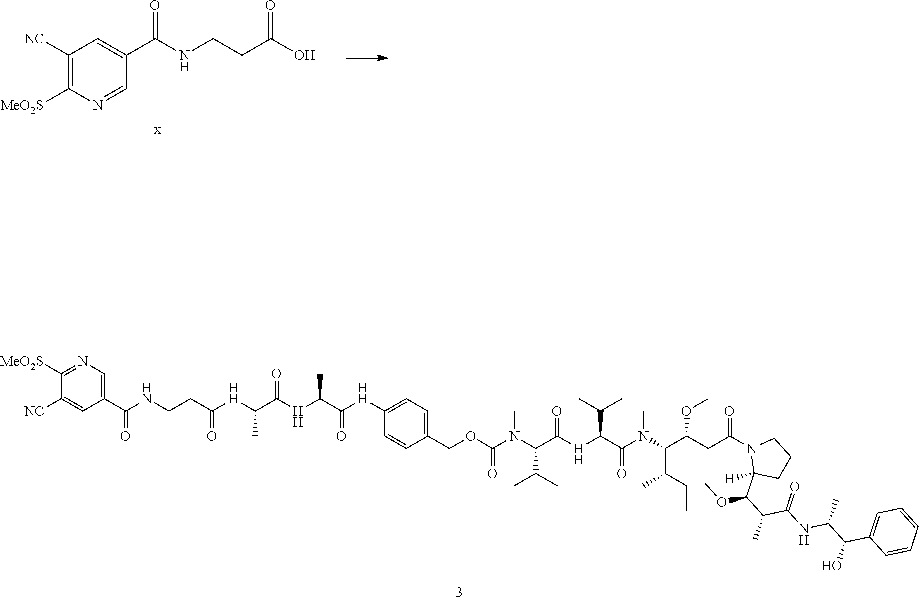

3-(5-Cyano-6-(methylsulfonyl)nicotinamido)propanoic acid (x) (0.027 g, 0.090 mmol) and HATU (0.037 g, 0.097 mmol) were dissolved in 0.4 mL of DMF and stirred for 10 minutes. Then xiv was added dropwise followed by DIEA (0.034 ml, 0.19 mmol). The reaction was stirred for 1.5 hours and then purified using reverse phase chromatography (Waters CSH-C18 Column, 19×250 mm×5 um, 35-70% acetonitrile in water (with 0.1% formic acid as modifier) to provide 4-((S)-2-((S)-2-(3-(5-cyano-6-(methylsulfonyl)nicotinamido)propanamido)propanamido)propanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (3). LC-MS: (ESI, m/z): [M+H]⁺=1288.665. ¹H NMR (600 MHz, CD₃CN) δ 9.12 (s, 1H), 8.68 (s, 1H), 8.63 (s, 1H), 7.66 (d, J=8.4Hz, 2H), 7.4-7.06 (m, 9H) 6.66 (d, J=8.5Hz, 1H), 6.55 (d, J=7.75 Hz, 1H), 5.18 (d, J=12.18 Hz, 1H), 5.04 (d, J=12.18 Hz, 1H), 4.71 (d, J=4.16 Hz, 1H), , 4.63 (m, 1H), 4.45 (q, J=7.3Hz, 1H), 4.32-4.00 (m, 4H), 3.9-3.73 (m, 3H), 3.67 (m, 2H), 3.58-3.38 (m, 3H), 3.36 (s, 3H), 3.35 (s, 3H), 3.28 (3, 3H), 3.18 (m, 1H), 3.01 (s, 3H) 2.86(b, 3H), 2.63 (m, 1H), 2.52 (m, 1H), 2.46 (b, 2H), 2.17 (b, 2H) 1.99 (m, 1H) 1.9-1.6 (m, 4H), 1.59 (b, 1H), 1.48 (d, J=7.38 Hz, 3H)1.36 (m, 4H), 1.12 (d, J=6.81 Hz, 3H), 1.02 (d, J=6.95 Hz, 3H), 0.98 (m, 4H) 0.92-0.67 (m, 15H).

### Example 4—Conjugation Protocols

Protocol 1: Sacituzumab with heavy chain engineered Cys residue (S375C-two heavy chains) was decapped using a literature procedure (WO2017072662, incorporated herein by reference) and diluted to 5 mg/mL with PBS. DMSO was added to the monoclonal antibody (mAb) solution to arrive at a 90% buffer 10% DMSO (v/v) solution. A 10 mM solution of Example 1 (3.1 Eq) in DMSO was added and the solution was mixed at rt for 2 hrs. The resulting ADC was purified via AKTA™ (desalting column, histidine pH 6.5 buffer, monitoring at 280 nm) and was characterized by LCMS (Agilent PLRP-S column, 1000 Å, 5 μm, 15-90% MeCN/H₂O with 0.1% formic acid, 80° C. column temperature) and SEC (Acquity UPLC Protein BEH SEC, 200 Å, 1.7 μm, 100 mM sodium phosphate, 200 mM NaCl, 0.02% azide, 5% IPA added to mobile phase for hydrophobic ADCs).

Protocol 2: Sacituzumab with heavy chain engineered Cys residue residue (S375C) was decapped using a literature procedure (WO2017072662) and diluted to 5 mg/mL with PBS. The antibody (20 mg) was exchanged into 40 mM Tris-Acetate, 1 mM EDTA, pH 8.3. The mAb was diluted to ˜10 mg/ml in a 90% buffer/10% DMF. A 10 mM solution of Example 3 (5.5 Eq) in DMF was added, and the solution was mixed at rt overnight. The ADC was purified via AKTA™ (desalting column, histidine pH 6.5 buffer, monitoring at 280 nm) and was characterized by LCMS (Agilent PLRP-S column, 1000 Å, 5 μm, 15-90% MeCN/H₂O with 0.1% formic acid, 80° C. column temperature) and SEC (Acquity UPLC Protein BEH SEC, 200 Å, 1.7 μm, 100 mM sodium phosphate, 200 mM NaCl, 0.02% azide, 5% IPA added to mobile phase for hydrophobic ADCs).

### ADC Data

The following Table 1 demonstrates average drug antibody ratio (DAR) and percent aggregation for ADCs utilizing the illustrative compounds of the present invention and the aforementioned conjugation protocol:

TABLE 1

| | Linker- | | | |
|---|---|---|---|---|
| ADC | Payload | Conjugation | Avg. | |
| Example | Example | Procedure | DAR | % Aggregation* |
| 4 | 1 | 1 | 1.9 | 0.70 |
| 5 | 2 | 1 | 1.8 | 0.98 |
| 6 | 3 | 2 | 1.8 | 1.3 |

### ADC N87 Cytotoxicity Assay Protocol

Illustrative ADCs of the present disclosure were subjected to a cell-based cytotoxicity assay (NCI-N87 cells) utilizing the following protocol:
Step 1: Seed 384-well Plates for Assay (45 uL per well) on day 0 Cells (NCI-N87) were quickly thawed in a cryo-vial by incubating them in a 37° C. water bath for <1 min until there is just a small bit of ice left in the vial. The vial was promptly removed and wiped down with 70% ethanol. The cells were transferred from the vial to a sterile centrifuge tube containing 8 mL of pre-warmed cell culture medium (RPMI-1640 (Cat #30-2001)+10% FBS+1% P/S). The vial was flushed with an additional 1 mL of medium to ensure complete transfer of cells to the centrifuge tube. The cells were then centrifuged at 150 g for 5 minutes. The supernatant was aspirated, and the cell pellet was resuspended in 10-20 mL cell culture medium (RPMI-1640 (Cat #30-2001)+10% FBS+1% P/S). Cells were counted using Vi-cell and prepared 1,500 cells/45 uL per well. Then added 45 uL/well of cells into Corning® 384-well Low Flange White Flat Bottom Polystyrene TC-treated Microplates (Corning, Cat #3570) using Standard Cassette Combi (if needed, dispense 1 dummy plate at 20 uL to help normalize Combi, using medium speed). The plates were spun down at 150 g for 30 seconds.
Step 2: Add ADCs on day 1. The ADC vials and reference stock were taken out and allowed to thaw at RT. The tubes were centrifuged at 2000 g for 30 seconds. The 10× Intermediate assay plates (Waters plate, Cat #186002632) were prepared using a Bravo liquid handler. Serial dilutions were made using a buffer of 10 mM pH 6.5 histidine 9% sucrose buffer. Media (no cells) was used for Max_E. Then added 5 uL of 10× stock from intermediate plate to assay plate using a Bravo liquid handler using a very slow speed so the cell monolayer wasn't disturbed. The plates were spun down at 150 g for 30 seconds.
   Step 3: CellTiter-Glo 2.0 Assay (Promega, Cat #G9242) on day 7 (CellTiter-Glo kit stored at −70° C.) The CellTiter-Glo® 2.0 Reagent was thawed at 4° C. overnight (reagent was not exposed to temperatures above 25° C.) The kit was equilibrated to RT for approximately 30 minutes. CellTiter-Glo® 2.0 Reagent (20 uL) was added to 50 uL of medium containing cells using Standard Cassette Combi. The contents were mixed for 2-3 minutes on an orbital shaker to induce cell lysis. The plates were spun down at 150 g for 30 seconds. The plates were allowed to incubate at RT for 5 minutes to stabilize the luminescent signal. The luminescence was recorded to calculate an EC₅₀ value, using an integration time of 0.25-1 second per well as a guideline. Illustrative ADCs of the present disclosure were tested in the above NCI-N87 cytotoxicity assay, and results are provided in Table 2 below:

TABLE 2

| ADC | |
|---|---|
| Example | EC₅₀ (nM) |
| 4 | 0.28 |
| 5 | 0.40 |
| 6 | 0.44 |

It will be appreciated that various of the above-discussed and other features and functions, or alternatives thereof, may be desirably combined into many other different systems or applications. Various presently unforeseen or unanticipated alternatives, modifications, variations or improvements therein that may be subsequently made by those skilled in the art are also intended to be encompassed by the following claims.

What is claimed:

1. A compound of Formula I, or a pharmaceutically acceptable salt, solvate, or stereoisomer thereof: 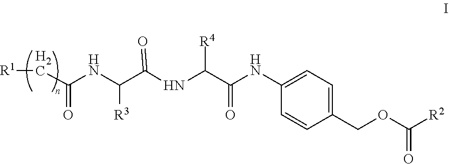
wherein:
R¹ is selected from: 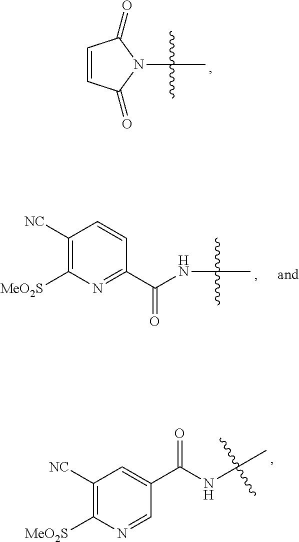 [SMILES: *N1C(=O)C=CC1=O ; *NC(=O)c1ccc(C#N)c(S(C)(=O)=O)n1 ; *NC(=O)c1cnc(S(C)(=O)=O)c(C#N)c1] [symbol]
wavy line indicates the site of covalent attachment;
R² is a cytotoxic drug;
R³ and R⁴ independently represent C₁₋₃ alkyl or a naturally occurring or unnatural amino acid acid side chain; and
n is an integer from 1 to 4.

2. The compound according to claim 1 wherein R¹ is

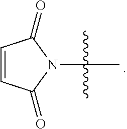 [SMILES: *N1C(=O)C=CC1=O]

3. The compound according to claim 1 wherein R¹ is

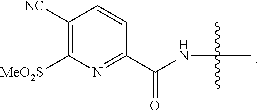 [SMILES: *NC(=O)c1ccc(C#N)c(S(C)(=O)=O)n1]

4. The compound according to claim 1 wherein R¹ is

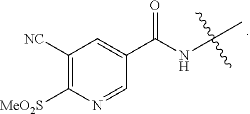 [SMILES: *NC(=O)c1cnc(S(C)(=O)=O)c(C#N)c1]

5. The compound according to claim 1 wherein R² is a cytotoxic drug selected from anthracyclines, auristatins, camptothecins, duocarmycins, etoposides, maytansinoids, pyrrolobenzodiazepine dimers, DNA minor groove binders, taxanes, vinca alkaloids, enediynes, anti-tubulins, and vinca alkaloids.

6. The compound according to claim 5 wherein R² is selected from auristatin T, auristatin E, auristatin F phenylenediamine, benzolyl-aurstatin E ester, 5-benzoylvaleric acid-AE ester, monomethyl auristatin F, lipophilic monomethyl aurstatin F, monomethyl auristatin E, lexitropsins, duocarmycins, paclitaxel and docetaxel, T67 (Tularik), vincristine, vinblastine, vindesine, vinorelbine, nicotinamide phosphoribosyltranferase inhibitor (NAMPTi), tubulysin M, doxorubicin, morpholino-doxorubicin, cyanomorpholino-doxorubicin, melphalan, methotrexate, mitomycin C, etoposide, CC-1065 analogue, calicheamicin, maytansine, an analog of dolastatin 10, rhizoxin, palytoxin, baccatin derivatives, taxane analogs (e.g., epothilone A and B), nocodazole, colchicine and colcimid, estramustine, cryptophysins, cemadotin, maytansinoids, combretastatins, discodermoide, tesirine and eleuthrobin.

7. The compound according to claim 1 wherein R² is an auristatin drug.

8. The compound according to claim 7 wherein R² is an auristatin drug selected from auristatin E auristatin F phenylenediamine, benzolyl-aurstatin E ester, 5-benzoylvaleric acid-auristatin E ester, monomethyl aurstatin F, and monomethyl auristatin E.

9. The compound according to claim 1 wherein R² is monomethyl auristatin E.

10. The compound according to claim 1 wherein R² is a pyrrolobenzodiazepine dimer.

11. The compound according to claim 1 wherein R³ and R⁴ are independently selected from C₁₋₃ alkyl.

12. The compound according to claim 1 wherein R³ and R⁴ are both CH₃.

13. The compound according to claim 1 wherein R³ and R⁴ are independently a naturally occurring or non-naturally occurring amino acid residue.

14. The compound according to claim 1 and pharmaceutically acceptable salts, solvates, or stereoisomer thereof, comprising a structure of Formula II: 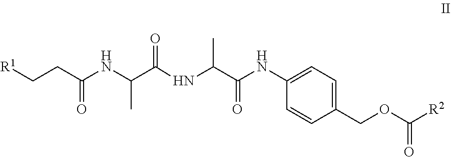
wherein R¹ and R² are as described herein.

15. The compound according to claim 14 wherein R² is an auristatin drug selected from auristatin E, auristatin F phenylenediamine, benzolyl-aurstatin E ester, 5-benzoylvaleric acid-AE ester, monomethyl aurstatin F, monomethyl auristatin E, or a pyrrolobenzodiazepine dimer.

16. A compound of Formula III or a pharmaceutically acceptable salt, solvate, or stereoisomer thereof. 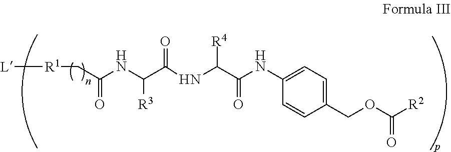
wherein
R¹ is selected from: 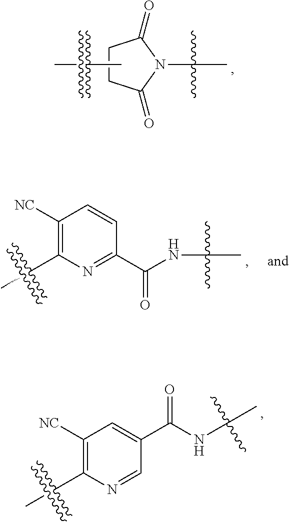 [symbol]
single wavy line indicates the site of covalent attachment to —(CH₂)ₙ; [symbol]
double wavy line indicates the site of covelant attachment to a sulfur of a cysteine residue of L′
R² is a cytotoxic drug;
R³ and R⁴ independently represent C₁₋₃ alkyl or a naturally occurring or unnatural amino acid acid side chain;
n is an integer from 1 to 4;
L′ is a ligand that is an antibody, or an antigen-binding fragment of an antibody); and
p is a positive rational number from 1 to 24, including fractions and decimals.

17. The compound according to claim 16 wherein and wherein R² is an auristatin drug selected from aurstatin E, auristatin F phenylenediamine, benzolyl-aurstatin E ester, 5-benzoylvaleric acid-AE ester, monomethyl aurstatin F, monomethyl auristatin E, or a pyrrolobenzodiazepine dimer, and R³ and R⁴ both are CH₃.

18. The compound according to claim 16 wherein R² is monomethyl auristatin E.

19. The compound according to claim 1, or a pharmaceutically acceptable salt thereof which is

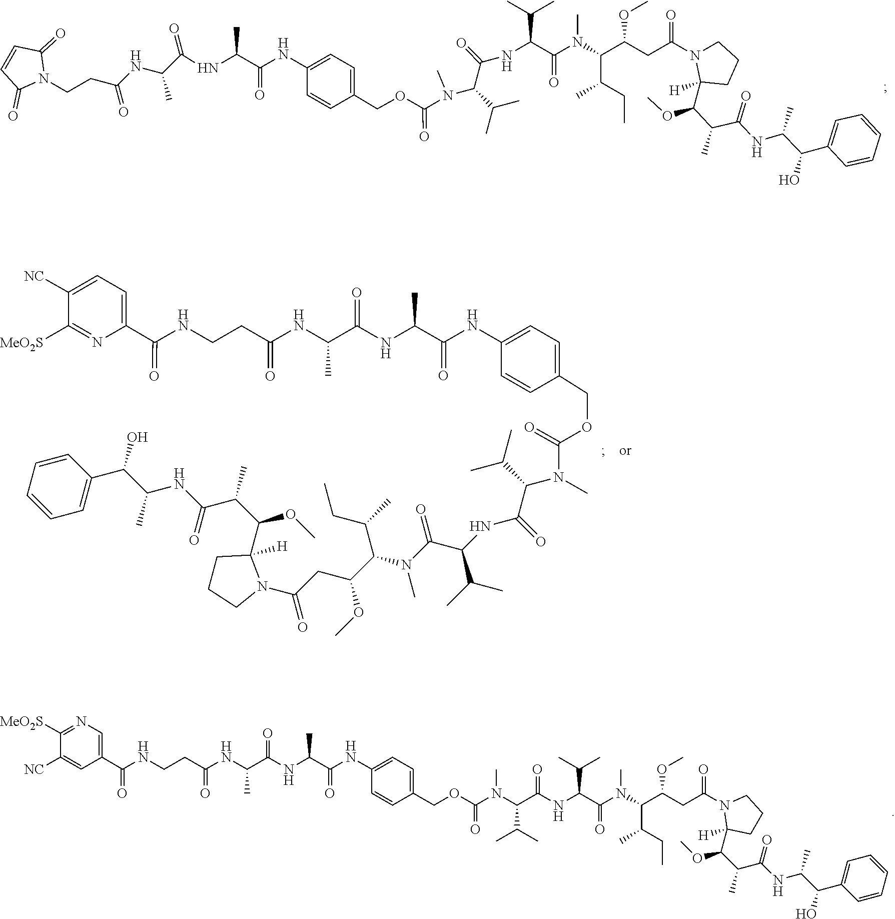

20. The compound according to claim 16, or pharmaceutically acceptable salts, or solvates, selected from: 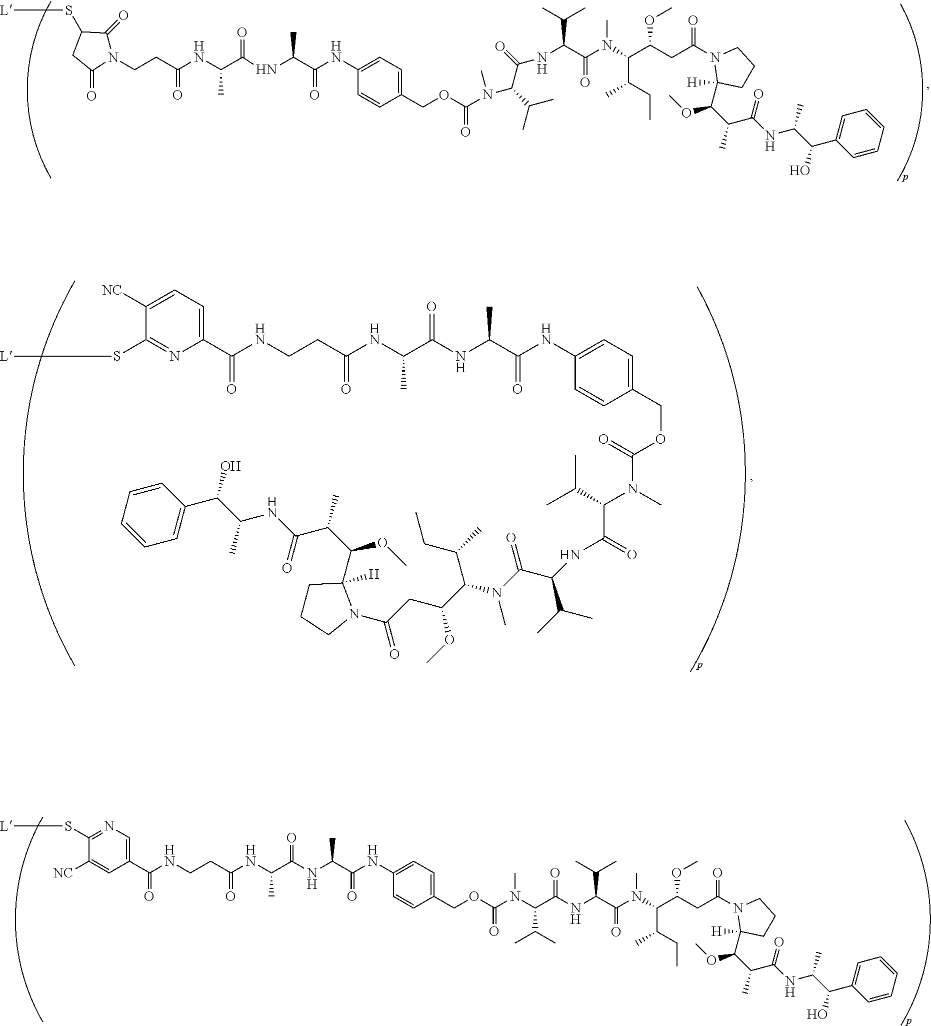
wherein L′ is an antibody and p is an integer from 1 to 8.

21. The compound according to claim 20, or pharmaceutically acceptable salts, or solvates, thereof wherein p is an integer from 1 to 2.

22. A composition comprising a compound of claim 1, or a pharmaceutically acceptable salt or solvate thereof, and a pharmaceutically acceptable carrier.

23. A pharmaceutical composition comprising a compound of claim 16, or a pharmaceutically acceptable salt or solvate thereof, and a pharmaceutically acceptable carrier.

24. A method of treating or preventing a cancer selected from breast cancer, ovarian cancer, cervical cancer, uterine cancer, prostate cancer, kidney cancer, urethral cancer, bladder cancer, liver cancer, stomach cancer, endometrial cancer, salivary gland cancer, esophageal cancer, melanoma, glioma, neuroblastoma, sarcoma, lung cancer (for example, small cell lung cancer and non-small cell lung cancer) colon cancer, rectal cancer, colorectal cancer, leukemia, acute promyelocytic leukemia, chronic myeloid leukemia, chronic lymphocytic leukemia), bone cancer, skin cancer, thyroid cancer, pancreatic cancer, and lymphoma (for example, Hodgkin's lymphoma, non-Hodgkin's lymphoma, or recurrent anaplastic large cell lymphoma) in a subject in need thereof, said method comprising administering to a subject in need of such treatment an effective amount of a compound of claim 16, or a pharmaceutically acceptable salt or solvate thereof, or a pharmaceutical composition comprising said compound, salt or solvate thereof.

25. A method for treating and/or preventing a tumor, comprising administering to a patient in need thereof an effective amount of the compound or pharmaceutical composition comprising the compound of claim 16.
